(19) United States
(12) Patent Application Publication
(10) Pub. No.: US 2012/0271331 A1
(43) Pub. Date: Oct. 25, 2012

(54) DEVICES AND METHODS FOR ANCHORING TISSUE

(75) Inventors: John TO, Newark, CA (US); Niel F. STARKSEN, Los Altos Hills, CA (US); Mariel FABRO, San Jose, CA (US); Nathan B. PLIAM, Los Altos Hills, CA (US); Karl S. IM, Burlingame, CA (US)

(73) Assignee: Guided Delivery Systems Inc., Santa Clara, CA (US)

(21) Appl. No.: 13/540,499

(22) Filed: Jul. 2, 2012

Related U.S. Application Data
(63) Continuation of application No. 11/202,474, filed on Aug. 11, 2005.
Application No. 11/202,474 is a continuation-in-part of application No. 10/792,681, filed on Mar. 2, 2004.
Application No. 10/792,681 is a continuation-in-part of application No. 10/741,130, filed on Dec. 19, 2003.

(51) Int. Cl. A61B 17/03

(52) U.S. Cl. 606/151

(57) ABSTRACT

Anchors, anchoring systems, anchor delivery devices, and method of using anchors are described. An anchor may be a flexible anchor having two curved legs that cross in a single turning direction to form a loop, wherein the legs are adapted to penetrate tissue. The ends of the curved legs may be blunt or sharp. The anchor can assume different configurations such as a deployed configuration and a delivery configuration, and the anchor may switch between these different configurations. In operation, the anchor may be inserted into tissue by releasing the anchor from a delivery configuration so that the anchor self-expands into the deployed configuration, so that the legs of the anchor may penetrate the tissue in a curved pathway.

### CROSS REFERENCE TO RELATED APPLICATIONS

The present application is a continuation of U.S. patent application Ser. No. 11/202,474, filed Aug. 11, 2005, which is a continuation-in-part of U.S. patent application Ser. No. 10/792,681, filed Mar. 2, 2004, which is a continuation-in-part of U.S. patent application Ser. No. 10/741,130, filed Dec. 19, 2003. The full disclosures of these applications are hereby incorporated by reference in their entirety. This present application is also related to U.S. patent application titled “METHODS AND DEVICES FOR DEPLOYMENT OF TISSUE ANCHORS” by John To, Niel Starksen, Mariel Fabro, Nathan Pliam, and Karl Im, filed Aug. 10, 2005.

### TECHNICAL FIELD

The devices and methods described herein relate generally to the field of surgery and more particularly to devices for anchoring tissue and/or anchoring materials to tissue, and to methods of using these devices.

### BACKGROUND

Anchors may be used to join tissues or to attach material to tissue. Tissues may be joined to close wounds, to modify body structures or passages, or to transplant or graft tissues within the body. For example, anchors may be used to close both internal and external wounds such as hernias. Implants and grafts may also be attached to tissue with anchors. Typical grafts include autograft and allograft tissue, such as a graft blood vessels, dermal (skin) grafts, corneal grafts, musculoskeletal grafts, cardiac valve grafts, and tendon grafts. In addition to tissue grafts, virtually any material or device may be implanted and attached within a body using anchors, including pacemakers, stents, artificial valves, insulin pumps, etc. Anchors may also be used to stabilize tissue relative to other tissues, or to stabilize a graft or implant against a tissue.

Traditional anchors used in surgery include clips, staples, or sutures, and may also be referred to as tissue anchors. These devices are usually made of a biocompatible material (or are coated with a biocompatible material), so that they can be safely implanted into the body. Most tissue anchors secure the tissue by impaling it with one or more posts or legs that are bent or crimped to lock the tissue into position. Thus, most traditional anchors are rigid or are inflexibly attached to the tissue. However, rigid tissue attachments may damage the tissue, particularly tissues that undergo repetitive motions, such as muscle tissue. For example, when a tissue with an attached anchor moves, the tissue may pull against the inflexible anchor, tearing the tissue or dislodging the anchor from the tissue. This problem may be exacerbated when the anchors are left in the tissue for long periods of time.

Most tissue anchors require an applicator. In particular, traditional anchors require an applicator to apply force to drive the anchor into the tissue. Furthermore an applicator may also be necessary to lock the anchor in the tissue once it has been inserted. For example, the applicator may crimp or deform the anchor so that it remains attached in the tissue and secures the graft or implant against the tissue. Such applicators may be difficult to use, particularly in small spaces or when the tissue to be operated on is located in hard to reach regions of the body. In some cases, the anchor itself may be difficult to maneuver in such locations, because it may be too large.

The size and maneuverability of the applicator and the anchor are particularly important when the anchors will be used for minimally invasive procedures such as laproscopic or endoscopic procedures. Minimally invasive surgery allows physicians to perform surgical procedures resulting in less pain and less recovery time than conventional surgeries. Laparoscopic and endoscopic procedures typically access the body through small incisions into which narrow devices (e.g., catheters) are inserted and guided to the region of the body to be operated upon. Anchors compatible for use with laproscopic and endoscopic procedures must be an appropriate size, and must also be manipulatable through a catheter or other instrumentation used for the laproscopic or endoscopic procedure.

Therefore, it would be beneficial to have improved anchor devices, methods and systems for joining tissue to tissue or joining tissues to implants or grafts. Ideally, such devices would be appropriately flexible to prevent damage to the tissue when it is repetitively loaded. Additionally, such devices would be useful and appropriate for laproscopic and endoscopic applications. At least some of these objectives will be met by the present invention.

### DESCRIPTION OF THE BACKGROUND ART

Published U.S. Application 2003/0033006 describes a device for the repair of arteries. Other U.S. patents of interest include: U.S. Pat. No. 4,014,492, U.S. Pat. No. 4,043,504, U.S. Pat. No. 5,366,479, U.S. Pat. No. 5,472,004, U.S. Pat. No. 6,074,401, U.S. Pat. No. 6,149,658, U.S. Pat. No. 6,514,265, U.S. Pat. No. 6,613,059, U.S. Pat. No. 6,641,593, U.S. Pat. No. 6,607,541, and U.S. Pat. No. 6,551,332. Other U.S. patent applications of interest include: U.S. 2003/0199974, and U.S. 2003/0074012. All of the above cited patents and applications are hereby incorporated by reference in the present application.

Other patent applications of interest include: U.S. patent application Ser. No. 10/656,797 (titled, “DEVICES AND METHODS FOR CARDIAC ANNULUS STABILIZATION AND TREATMENT”), filed on Sep. 4, 2003, and Ser. No. 10/461,043 (titled, “DEVICES AND METHODS FOR HEART VALVE REPAIR”), filed on Jun. 13, 2003, the latter of which claims the benefit of U.S. Provisional Patent Applications No. 60/388,935 (titled “METHOD AND APPARATUS FOR MITRAL VALVE REPAIR”), filed on Jun. 13, 2002; No. 60/429,288 (titled “METHODS AND DEVICES FOR MITRAL VALVE REPAIR”), filed on Nov. 25, 2002; No. 60/462,502 (titled, “HEART SURGERY INTRODUCER DEVICE AND METHOD”), filed on Apr. 10, 2003; and No. 60/445,890 (titled “METHODS AND DEVICES FOR MITRAL VALVE REPAIR”), filed on Feb. 6, 2003. The full disclosures of all of the above-listed patent applications are herby incorporated by reference.

### BRIEF SUMMARY OF THE INVENTION

Described herein are flexible anchors, anchoring systems, and methods of using flexible anchors. In some variations, a flexible anchor comprises two curved legs crossing in a single turning direction to form a loop, wherein the legs are adapted to penetrate tissue. For example, the ends of the curved legs may be blunt (and still capable of penetrating tissue), or they may be sharp. The ends of the legs may also be beveled. The anchor may be made out of any appropriate material. For example, the anchor may be made from a shape-memory material such as a Nickel-Titanium Alloy (Nitinol). In some variations, the anchor is made of an elastic or a superelastic material. The entire anchor may be made from the same material, or the anchor may have regions that are made from different materials. In some variations, different regions of the anchor may have different properties (including elasticity, stiffness, etc.).

In some variations, the anchor can assume different configurations, and the anchor may switch between these different configurations. For example, the anchor may have a delivery configuration in which the legs are collapsed, and a deployed configuration in which the legs are expanded. In operation, the anchor may be inserted into tissue by releasing the anchor from a delivery configuration so that the anchor self-expands into the deployed configuration. As the anchor is deployed, the legs of the anchor may penetrate the tissue in a curved pathway.

In some variations, the ratio of the spacing between the legs (e.g., the ends of the legs) in the delivery configuration (at their narrowest separation) to the spacing between the leg ends in the deployed configuration (at their widest separation) is about 1:2 to about 1:20. In some variations, this ratio of the spacing between the legs is between about 1:8 and about 1:9. Thus, when the anchor is deployed, the legs are spread out within the tissue, distributing the forces from the anchor across the tissue. When the anchor is located in the tissue, the anchor absorbs energy during dynamic loading of the tissue to relieve peak stresses on the tissue. In some variations, the elasticity of the anchor is about half to about five times the elasticity of the tissue into which the anchor is to be inserted. When the anchor has been deployed in a tissue, the anchor may expand or collapse from the deployed configuration to absorb energy during dynamic loading of the tissue.

Flexible anchors for insertion into a tissue may have two legs that cross in a single turning direction to form a loop, and may also have a deployed configuration wherein, when the anchor is inserted into tissue, the anchor absorbs energy during repetitive loading of the tissue to relieve peak stresses on the tissue by collapsing or expanding from the deployed configuration. The anchor may also have a delivery configuration in which the legs are collapsed.

In general, the anchor has a single turning direction, so that from the tip of one leg of the anchor to the tip of the other leg of the anchor, the anchor curves or bends only in a single turning direction (e.g., to the right or to the left). Thus, the legs and the loop region of the anchor all have only a single turning direction. The legs (e.g., the ends of the legs) of the anchor typically penetrate tissue in a curved path, and in opposing directions that minimize tissue deflection. In some variations, the leg ends are expanded to deploy the anchor into tissue so that the expansion of the leg ends drives the anchor into the tissue.

Also described herein are methods of attaching an anchor to tissue. The methods may include releasing an anchor from a delivery configuration, where the anchor has two legs adapted to penetrate tissue, and the legs cross in a single turning direction to form a loop. The legs are collapsed in the delivery configuration so that releasing the anchor from the delivery configuration deploys the legs through the tissue in a curved path to secure the anchor against the tissue. The method may also include the step of compressing the anchor into the delivery configuration. In some variations, an implant (e.g., a graft, a suture, etc.) may be secured to the tissue by the anchor. For example, the anchor may penetrate the implant and the tissue, or the implant may be secured to an anchor that penetrates the tissue.

These and other aspects and variations are described more fully below with reference to the drawings.

### BRIEF DESCRIPTION OF THE DRAWINGS

FIG. 1 is a cross-sectional view of a heart with a flexible anchor delivery device being positioned for treatment of a mitral valve annulus;

FIGS. 2A and 2B are cross-sectional views of a portion of a heart, schematically showing positioning of a flexible device for treatment of a mitral valve annulus;

FIGS. 2C and 2D are cross-sectional views of a portion of a heart, showing positioning of a flexible anchor delivery device for treatment of a mitral valve annulus;

FIG. 3 is a perspective view of a distal portion of an anchor delivery device;

FIG. 4. is a perspective view of a segment of a distal portion of an anchor delivery device, with anchors in an un-deployed shape and position;

FIG. 5 is a different perspective view of the segment of the device shown in FIG. 4;

FIG. 6. is a perspective view of a segment of a distal portion of an anchor delivery device, with anchors in a deployed shape and position;

FIGS. 7A-7E are cross-sectional views of an anchor delivery device, illustrating a method for delivering anchors to valve annulus tissue;

FIGS. 8A and 8B are top-views of a plurality of anchors coupled to a self-deforming coupling member or “backbone,” with the backbone shown in an un-deployed shape and a deployed shape;

FIGS. 9A-9C are various perspective views of a distal portion of a flexible anchor delivery device;

FIGS. 10A-10F demonstrate a method for applying anchors to a valve annulus and cinching the anchors to tighten the annulus, using an anchor delivery device;

FIG. 11 shows a heart in cross-section with a guide catheter device advanced through the aorta into the left ventricle;

FIGS. 12A-12F demonstrate a method for advancing an anchor delivery device to a position for treating a heart valve;

FIGS. 13A and 13B are side cross-sectional views of a guide catheter device for facilitating positioning of an anchor delivery device;

FIG. 14 is a perspective view of an anchor as described herein;

FIGS. 15A and 15B show perspective views of the anchor of FIG. 14 in an expanded and compressed state, respectively; and

FIGS. 16A to 16C show an anchor begin deployed into tissue, as described herein.

FIGS. 17A and 17B show anchors as described herein.

### DETAILED DESCRIPTION

Included in this description are anchors including flexible anchors for securing to tissue. In some variations, devices, systems and methods including anchors are described for use in facilitating transvascular, minimally invasive and other “less invasive” surgical procedures, by facilitating the delivery of treatment devices at a treatment site. Although many of the examples described below focus on use of anchor devices and methods for mitral valve repair, these devices and methods may be used in any suitable procedure, both cardiac and non-cardiac.

### Anchors

An anchor may be any appropriate fastener. In particular, an anchor may be a flexible anchor having two curved legs that cross in a single turning direction to form a loop, wherein the legs are adapted to penetrate tissue. FIG. 14 illustrates one example of an anchor as described herein. In FIG. 14, the anchor 600 has curved legs 601, 602 and a loop region 605. The legs and loop region all have a single turning direction, indicated by the arrows 610.

The single turning direction describes the curvature of the legs and loop region of the anchor, including the transitions between the legs and loop region. For example, in FIG. 14 the limbs of the anchor and the loop region define a single direction of curvature when following the length of the anchor from tip to tip. Starting at the tip 612 of the lower leg 602 of the anchor shown in FIG. 14, the anchor curves only in one direction (e.g., to the right) from the tip of one leg of the anchor 612, through the loop region 605, to the tip of the other leg 614. Another way to describe the single turning direction of the anchor is to imagine a point traveling along the anchor from the tip of one leg to the tip of the other end. As the point moves along the length of the anchor down the legs and loop region, the point turns only one direction (e.g., right/left or clockwise/counterclockwise). The angle that the point turns (the turning angle, from which the point is deflected from continuing straight ahead) anywhere along the length of the anchor can be of any appropriate degree, i.e., between 0° and 180°. The anchor is generally continuously connected from leg-tip to leg-tip, as shown in FIG. 14.

Anchors having a single turning direction may bend or flex more than anchors having more than one turning direction. For example, anchors having more than one turning direction typically have one or more surfaces (e.g., abutment surfaces) that inhibit the collapse and/or expansion of the anchors, as described further below.

The anchor shown in FIG. 14 is in a deployed configuration, in which the legs of the anchor are expanded. The legs (which may also be referred to as arms) of this anchor 601, 602 are curved and thus form a semicircular or circular shape on either side of the loop region 605. The legs may be less uniformly curved, or un-curved. For example, the legs may form elliptical or semi-elliptical shapes, rather than circular/semicircular shapes. In some variations, the legs are not continuously curved, but may contain regions that are uncurved. In some variations, the anchor may comprise sharp bends.

The anchors described herein may have a deployed configuration and a delivery configuration. The deployed configuration is the configuration that the anchor assumes when it has been deployed into the tissue. The anchor may be relaxed in the deployed configuration. The delivery configuration is any configuration in which the anchor is prepared for delivery. In some variations, the arms are compressed in the delivery configuration, so that the anchor has a smaller or narrower profile. The narrower profile may allow the anchors to be delivered by a small bore catheter. For example, anchors in a delivery configuration may fit into a catheter having an I.D. of about 0.5 mm to about 3.0 mm. In some variations, the anchor may be used with a delivery device having an I.D. of about 1 mm.

The ends of the legs 612, 614 are configured to penetrate tissue, so that the legs of the anchor may pass into the tissue when the anchor is deployed, as described more fully below. In some variations, the leg ends are blunt, or rounded. Blunt or rounded ends may still penetrate tissue. In some variations, the tips of the leg ends are sharp, or pointed, as shown in FIG. 14. In FIG. 14, the leg ends are beveled so that they have a sharp end. In some variations, the ends of the legs may include one or more barbs or a hooked region (not shown) to further attach to the tissue.

The loop region 605 may also be referred to as an eye, eyelet or eye region. In the exemplary anchor shown in FIG. 14, the loop region comprises a single loop that is continuous with the legs 601, 602, and lies equally spaced between the two legs. For example, both legs 601, 602, cross once to form the loop region having a single loop. In some variations, the legs have different lengths or shapes, and the loop region is not centered between equal-sized legs. In some variations, the loop region has more than one loop. For example, the loop region may be formed by more than one complete turn. Thus the loop region may comprise a helical shape having more than one loop (e.g., two loops, three loops, etc.).

The loop region may be of any appropriate size, and may change size based on the configuration of the anchor. For example, when the anchor is in a deployed configuration, the loop region may be larger (e.g., wider) than when the anchor is in a delivery configuration. In some variations, the loop region is smaller when the anchor is in a collapsed configuration, thus, the loop region may be of any appropriate shape, and may also change shape based on the configuration of the anchor. For example, the loop region may be more elliptical (e.g., narrower) in a delivery configuration, or more rounded.

The position of the legs may be changed depending on the configuration of the anchor. For example, the legs may be expanded or collapsed. The legs 601, 602 may contact each other by meeting at a point of contact 630. In some variations, the legs 601, 602 cross each other without contacting. In some variations, the legs contact each other, so that the loop 605 is a closed region. In some variations, the legs are attached to each other at the point of contact 630. In some variations, one of the legs may pass through a passage (e.g., a hole) in the other leg.

The anchor may also have a thickness. For example, the anchor shown in FIG. 14 is substantially planar, meaning that the legs typically move in a single plane (e.g., the plane parallel to the page). The anchor in FIG. 14 is formed of a substantially cylindrical wire-like member, and the anchor has a thickness that is approximately twice the thickness of the wire-like member, because the legs cross over each other at point 630. The legs or body of the anchor (including the loop region) may also be at least partially hollow. For example, the anchor may be formed from a tube, or may include a tube region. Thus, the anchor may include one or more hollow regions that may allow tissue ingrowth, or may be used to hold additional materials (e.g., drugs, electronics, etc.). In some variations, the hollow region of the anchor may comprise drugs that may be eluted. (e.g., time release drugs). Overall, the anchor may be of any appropriate thickness. Furthermore, in some variations, the legs may move in any appropriate direction, including directions that are different from the plane in which the legs lie. For example, in one variation, the legs move in a corkscrew fashion (e.g., from a delivery configuration to a deployed configuration).

In FIG. 14, the opening formed by the loop region creates a passage through the plane of the anchor, so that material (e.g., a tether) may pass through the loop, and therefore through the plane formed by the anchor legs and loop region. In this variation, the legs move mostly within this plane. In some variations, the anchor does not form a single plane as shown in FIG. 14, but instead, the legs extend in a single turning direction, and also extend up or down from the plane of the figure shown in FIG. 14. Furthermore, the loop region may also face a direction that is not parallel to the plane formed by the anchor. For example, the loop region may face a direction that is parallel to the plane formed by the legs. Thus, a material passing through the loop region may pass through in a direction that is not perpendicular to the plane formed by the rest of the anchor. The legs and/or the loop region may be twisted so that they extend from a plane that is not the same as the plane formed by the rest of the anchor.

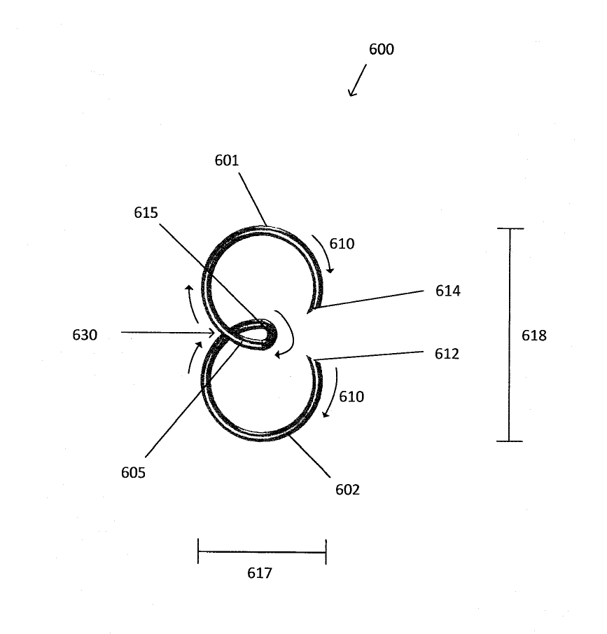

An anchor may be made of a single material, or it may be formed of many materials. In one variation, the anchor is made of a single piece of material. For example, the anchor may be formed from a linear material (e.g., a wire) that is formed into the desired shape (e.g., the deployed configuration). In some variations, the anchor is cut or etched from a sheet of material, (e.g., Nitinol). In some variations, the anchor includes different regions that are connected or joined together. These different regions may be made of the same material, or they may be made of different materials. The different regions may include regions having different physical or material properties, such as material strength, flexibility, ductability, elasticity, and the like. For example, the loop region of the anchor may comprise a material having a different (e.g., a decreased or increased) stiffness compared to the leg regions. In FIG. 14, part of the loop region 605 is a segment 615 that is joined to the segments forming the legs 601, 602. In this example, the central portion 615 of the loop region 605 is less flexible than the legs 601, 602, so that it is less likely to deform (e.g., requires more energy) than the adjacent leg regions, and may maintain an approximate shape (e.g., an elliptical shape, as shown in FIGS. 14 and 15A-15B) of the loop region.

An anchor may be made of (or may contain a region or coating of) a biodegradable or bioabsorbable material. Biodegradable portions of the anchor may allow time-controlled changes in the mechanical or biochemical properties of the anchor and in the interaction of the anchor with the tissue. For example, an outer layer of the anchor may dissolve over time, rendering the anchor thinner and more flexible. Thus, an anchor may be initially quite thick (e.g., providing an initial strength or stiffness), but after insertion into the tissue, the outer layer may dissolve or be removed, leaving the anchor more flexible, so that it can better match the tissue compliance.

In some variations, a region having an enhanced flexibility creates a spring or hinge region that can enhance or limit the overall flexibility of the anchor or a region of the anchor. This can, in turn, affect the ability of the anchor to change configurations between a deployed and a delivery configuration. As described further below, a hinge or spring region may be used to enhance the effectiveness of the anchor during cyclic (e.g., repetitive) loading of a tissue into which an anchor has been inserted.

### Anchor Configurations

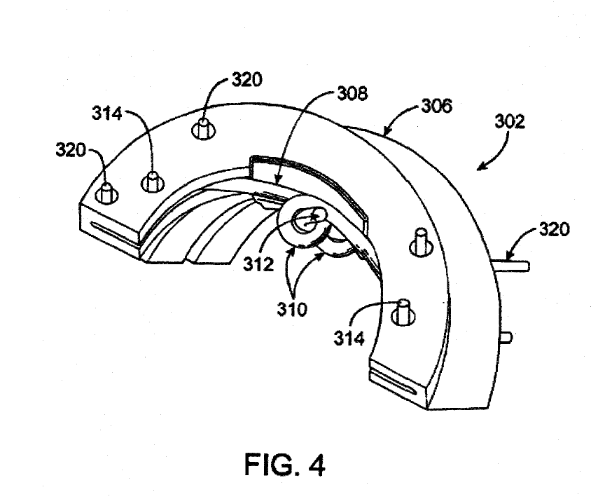

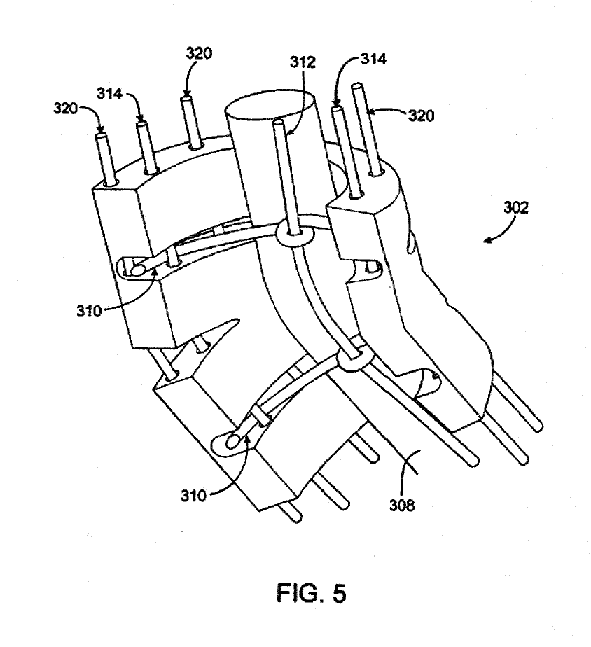

The anchors described herein are generally flexible anchors, and may transition between a deployed configuration and one or more compressed or expanded configurations. The deployed configuration may also be referred to as a relaxed configuration. As mentioned above, the delivery configuration may be a compressed configuration (as shown in FIG. 15B) or an expanded configuration (as shown in FIGS. 4 and 5). The anchor may by compressed or expanded to different amounts, so that there may be many expanded or compressed configurations.

FIGS. 15A and 15B show examples of an anchor in a deployed configuration and a delivery configuration, respectively. When the anchor is in the deployed configuration 650, as shown in FIG. 15A, the legs 601, 602 are typically expanded radially, and the loop region 605 has an opening 680 through which a material (e.g., a tether) may be attached or may pass. This deployed configuration is the configuration that this variation of the anchor assumes when external forces on the anchor are minimal.

At least a portion of the anchor comprises an elastic or superelastic material, such as a metal, alloy, polymer (e.g., rubber, poly-ether ether ketone (PEEK), polyester, nylon, etc.) or some combination thereof that is capable of elastically recovering from deformation. For example, the anchor may comprise a Nickel-Titanium Alloy (e.g., Nitinol), or a region that is a rubber or polymeric material. In some variations, the anchor may comprise a material having a shape memory. In some variations, the anchor may comprise a bioabsorbable and/or biodegradable material (e.g., polymers such as polylactic acid (polylactide), poly-lactic-co-glycolic acid (poly-lactido-co-glycolide), polycaprolactone, and shape memory polymers such as oligo(ε-caprolactone)diol and crystallisable oligo(ρ-dioxanone)diol, etc.).

When force is applied to the anchor, or to a tissue into which the anchor is embedded, the anchor may flex or bend and thereby absorb some of the energy applied, and change the configuration of the anchor. For example, the anchor may be compressed or expanded from a resting position. In particular, the anchor may be compressed from a deployed configuration such as the one shown in FIG. 15A into smaller delivery configuration such as the one shown in FIG. 15B.

In FIG. 15B, the anchor has been compressed into a delivery configuration by drawing the ends of the legs back so that the anchor has a smaller profile with a stored potential energy that can revert the anchor back into the deployed configuration (e.g., the anchor may be self-deforming). In this variation of the delivery configuration, the anchor profile is much narrower than in the deployed configuration. The legs of the anchor have been extended (reducing their curve), enlarging or expanding the opening formed by the loop region 605. In this example, the loop region remains narrow and elliptical, because one portion of the loop region 615 is less flexible than the other portions of the loop region and the leg regions, as described above. This less flexible portion of the loop, or loop size limiter 615, limits the width that the loop region may expand to, and comprises a sub-region of the loop region that is less flexible than other regions of the anchor (e.g., the legs). In some variations, the loop size limiter region is flexible. In some variations, the loop size limiter region comprises an inflexible material. In some variations, the loop region expands as the anchor (e.g., the anchor legs) is compressed into a delivery configuration, so that the overall size of the loop region increases both in width and length.

In some variations, the anchor has a delivery configuration in which the arms of the anchor are radially expanded from their position in the deployed configuration. FIGS. 4 and 5 illustrate an anchor with a delivery configuration having radially expanded arms, and FIG. 5 shows the corresponding deployed configuration for this anchor. The variation is discussed more fully in the “Examples” section below.

The anchor 600 may be compressed or expanded from the deployed configuration into a delivery configuration by any appropriate method. For example, the legs of the flexible anchor 601, 602 may be drawn back into the delivery configuration as shown in FIG. 15B, and held until the anchor is to be deployed into a tissue. Because the anchor comprises an elastic material, the anchor will typically store energy used to change the anchor from the delivery configuration to the deployed configuration. Upon releasing the anchor from the delivery configuration, the stored energy is released, and the anchor expands into the deployed configuration, as shown in FIG. 15A. When the anchor is compressed into a delivery configuration, this energy may be used to help drive the legs of the anchor into the tissue, and may draw the anchor into the tissue. Thus, the anchor may be self-expanding, self-deforming, or self-securing. In some variations, deployment of the anchor into the tissue drives the legs into tissue in a curved pathway, helping to pull and secure the anchor into the tissue, as described more fully below.

In FIGS. 15A and 15B, the deployed anchor has a much bigger leg span than the compressed anchor. In other words, the distance between the legs of the anchor in the deployed state 650 is larger than the distance between the legs of the anchor in the compressed state 660. In some variations, the ratio of the distance between the legs in the compressed state versus the distance between the legs in the deployed state is between about 1:2 to about 1:20. In some variations, the ratio of the distance between the legs in the compressed state versus the distance between the legs (e.g., at the ends of the legs) is between about 1:2 to about 1:10. In some variations, the ratio of the distance between the legs in the compressed state versus the distance between the legs (e.g., at the ends of the legs) is between about 1:8 to about 1:9. For example, the ratio of the distance between the legs in the compressed state of FIG. 15B versus the distance between the legs in the deployed state in FIG. 15A is approximately 1:6. The wide span of the deployed anchor may allow the anchor to distribute loading of the anchor over or wide area within the tissue matrix, preventing high local stresses on the tissue by distributing stresses on the tissue from the anchor over a larger area of the tissue. Distributing the forces over a larger area may prevent damage to the tissue, and may allow better attachment and healing. In general, higher stresses acting on a localized region of tissue may damage the tissue, potentially allowing the anchor to migrate and/or pull out of the tissue.

As described above, the material moduli, shapes and sizes of different regions of the anchor may be selected so that the compressed and/or expanded shape of the anchor may be controlled. For example, in FIG. 15B, the width of the compressed anchor is limited by the loop size limiter region 615 as described above. The forces required to compress or expand the anchor from the deployed configuration into the delivery configuration may be affected by the overall size and/or shape of the anchor, including the thickness of the legs and loop region.

As briefly described above, the anchor may be of any appropriate size or dimension. The anchor may have a width 617, length 618 and a thickness. For example, the length of the anchor may be measured as the span of the legs 618 as shown in FIG. 14. In one variation, the width of the anchor 617 in the deployed configuration may be less than 5 mm wide. In some variations, the anchor is between about 1 mm wide and about 9 wide in the deployed configuration. In some variations, the anchor is about 4 mm wide in the deployed configuration. Furthermore, the anchor may comprise any appropriate thickness or range of thicknesses. In some variations, the thickness of the anchor varies over the different regions (e.g., legs and loop region). In general, the anchor may comprise a thickness of between about 0.12 mm to about 0.75 mm. In one variation, the anchor is about 0.4 mm thick. In some variations, a portion of the loop region is thicker than a leg region of the anchor. For example, the loop size limiter region may be thicker than the leg regions, so that the leg regions are more readily bent than the loop region, as described above. The length 618 of the deployed anchor may be from about 1 mm to about 20 mm long. In some variations the deployed anchor is about 10 mm long.

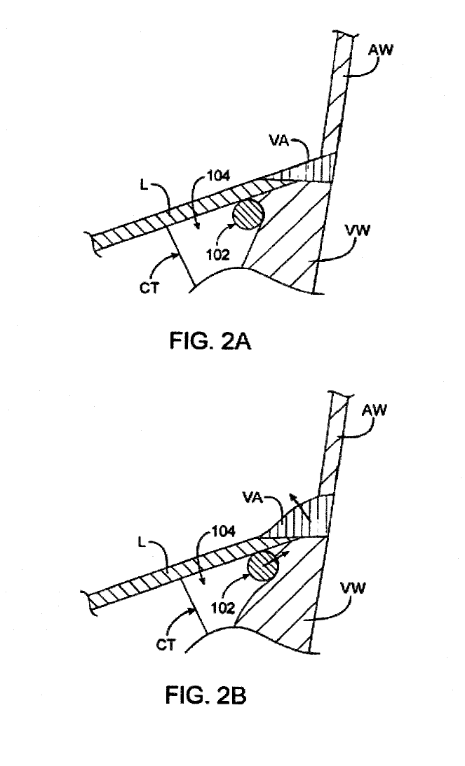

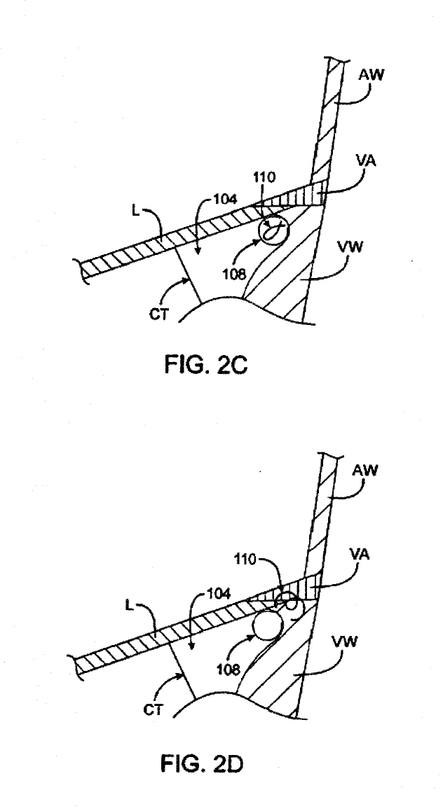

Anchors may be fabricated by any appropriate method. For example, an anchor may be made by working or shape-forming a material (e.g., an alloy or metal). In some variations, the anchor may be fabricated from a wire or wires. The examples of anchors shown in FIGS. 14 and 15 (as well as FIGS. 2-7 and 9-10) are all rounded, wire-like anchors. However, anchors may have flat or flattened sides. In some variations, the anchor or a part of the anchor is fabricated by cutting, stamping, or etching some or part of the anchor from a material. For example the anchor can be formed by cutting it out of a Nitinol sheet using a laser, EDM, or Photoetching. In some variations, the anchor or a part of the anchor is fabricated by molding or extrusion techniques. The entire anchor (e.g., legs and loop region) may be formed from a single continuous piece, or the anchor may be formed by attaching different component pieces together. Thus, an adhesive or other joining material may be used to connect different components of the anchor. The components may also be joined by welding, brazing or soldering.

Furthermore, an anchor may be treated or coated in any appropriate manner. In some variations, the anchor is sterilized. For example, an anchor may be irradiated, heated, or otherwise treated to sterilize the anchor. Sterilized anchors may be packaged to preserve sterility. In some variations, an anchor may be treated with a therapeutic material (e.g., a medicinal material such as an anti-inflammatory, an anticoagulant, an antiproliferative, a pro-proliferative, a thromboresistant material, a growth hormone, etc.) to promote healing. For example, the anchor may be coated with Vascular Endothelial Growth Factor (VegF), Fibroblast Growth Factor (FGF), Platelet-Derived Growth Factor (PDGF), Transforming Growth Factor Beta (TGFbeta, or analogs), insulin, insulin-like growth factors, estrogens, heparin, and/or Granulocyte Colony-Stimulating Factor (G-CSF). In some variations, the anchor may comprise pockets of material for release (e.g., medicinal materials). In some variations, the anchors may be coated with a material to promote adhesion (e.g., tissue cements, etc.) In some variations, the anchors may comprise a material to assist in visualizing the anchor. For example, the anchor may comprise a radiopaque material, or other contrast-enhancing agents (e.g., these agents may depend upon the material from which the anchor is made, and the imaging modality used). For example, the anchor may be coated with a metal, such as gold, aluminum, etc. The anchor may also comprise surface treatments, including texturing (e.g., by ion beam etching, photoetching, etc.), tempering (e.g., thermal or photo tempering), or the like. Additional examples of appropriate surface treatments may include electropolishing, chemical etching, grit or bead blasting, and tumbling in abrasive or polishing media. Polymer coatings may include Teflon or polyester (e.g., PET).

Coatings may be used to elute one or more drugs, as described above. For example, an outer layer may comprise a drug (or other dissolvable or removable layer) that exposes another layer (e.g., another drug layer) after it dissolves or is removed. Thus, the anchor may controllably deliver more than one drug in a controlled fashion. The release of a drug (or drug coating) may be affected by the geometry of the anchor, or the way in which the drug is arranged on or within the anchor. As described above, the anchor may comprise a hollow region or other regions from which a drug could be eluted. Thus, the anchor may include pits, slots, bumps, holes, etc. for elution of drugs, or to allow tissue ingrowth.

Different regions of the anchor may comprise different coatings. For example, the loop (or a portion of the loop) may include a lubricious coating, particularly in the region where the legs cross each other to form the loop. A lubricious coating (e.g., polytetrafluoroethylene (Teflon), silicones, hydrophilic lubricious coatings, etc.) in this region may help minimize friction when deploying the anchor and may give the anchor greater momentum during deployment.

Anchors may also include one or more sensors and/or telemetry for communicating with other devices. For example, an anchor may include sensors for sensing electrical potential, current, stress, strain, ion concentration, or for the detection of other compounds (e.g., glucose, urea, toxins, etc.). Thus, an anchor may include circuitry (e.g., microcircuitry) that may be powered by an on-board power source (e.g., battery) or by externally applied power (e.g., electromagnetic induction, etc.). Circuitry may also be used to analyze data. In some variations, the anchor may comprise telemetry (e.g., wireless telemetry) for sending or receiving data or instructions from a source external to the anchor. For example, the anchor may send data from a sensor to a receiver that is external to the subject. In some variations, the anchor may be used to controllably release material (e.g., drugs) into the tissue.

The anchor may also include one or more electrodes. Electrodes (e.g., microelectrodes) may be used to stimulate, or record from the tissue into which the anchor has been inserted. Thus, the anchor may be used to record electrical activity (e.g., cardiac electrical activity, muscle electrical activity, neuronal electrical activity, etc.). In some variations, the anchor can apply electrical stimulation to the tissue through the electrode. Stimulation or recording electrical activity may also be controlled either remotely (e.g., through telemetry) or by logic (e.g., control logic) on the anchor.

For example, the anchor may be deployed in nerves or other electrically active tissue so that electromagnetic or electrophysiological signals can be received or transmitted. In one variation, electrical signals are transmitted to a subject from (or through) an anchor for pain management or control. In one variation, the anchors may transmit signals to help control limp muscles (e.g., in stroke patients). Thus, an anchor may itself be an electrode. In one variation, an anchor is deployed into a tumor and energy (e.g., electrical energy) is applied through the anchor to ablate the tumor.

The anchors described herein may also include additional tissue-engaging features to help secure the anchors within the tissue, implant or graft. The anchors may include features to increase friction on the surface of the anchors, to capture tissue, or to restrict movement of the anchor and prevent pullout of the anchor.

For example, as described above, the ends of the anchor may comprise one or more barbs or hooks. In some variations, regions other than the ends of the legs (e.g., the body of the legs or loop region) may also include barbs or hooks for gripping. In one variation, a single curve having a tight radius may be present at the end of one or more of the anchor legs. The bend may hook into the tissue at the end of the leg like a long narrow fishhook.

Thus, the anchor may include regions of increased friction. In addition to the barbs described above, the anchor may also include tines, pores, holes, cut outs, or kinks. These features may increase friction and resistance to pullout, and (as described above) may also allow ingrowth of tissue that inhibits withdrawal of the anchor. The surface of the anchor may also be coated or textured to reduce friction or to increase interaction between the anchor and the tissue, implant, or other material.

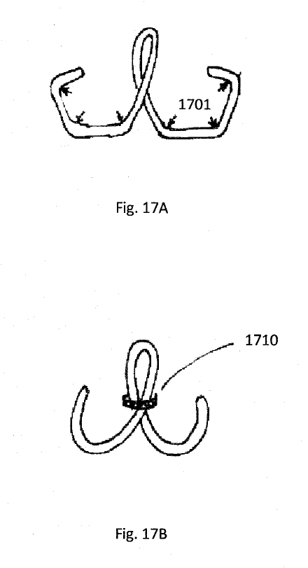

Movement of the anchor may also be restricted (or guided) to enhance attachment with tissue or other materials. For example, although the anchor typically curves in a single turning direction, the radius of the single turning direction may vary over the length of the anchor. In general, the tighter the bend radius of a region of the anchor, the greater the resistance to unbending. For example, the anchor may incorporate one or more bends that have a smaller radius of curvature (e.g., is a tighter bend) than other regions of the anchor. In one variation, the anchor comprises a plurality of relatively straight segments with intermediate, tight radius bends, as shown in FIG. 17A. The cumulative force required to unbend the plurality of tight bends 1701 of the legs may be greater than the force required to unbend the legs of a similar anchor having a single large radius of curvature (or a more continuously varying radius of curvature).

The loop region of the anchor may also be constrained. For example, the loop region of the anchor may be constrained in the deployed configuration or in the delivery configuration by a constraining member. Thus, the anchor may include a constraining member (e.g., a belt, band, sleeve, etc.) that constrains movement of the loop. The constraining member may be positioned on the anchor (e.g., at the crossover portion of the loop), and can lock the loop in a given size, shape, or position. The constraining member may prevent proximal flexure of the loop. FIG. 17B shows an example of a constraining member 1710 on an anchor. The constraining member may be adjustable. A constraining member may also constrain movement of a leg or legs of the anchor.

### Operation of the Anchor

The anchors described herein may be used as part of any appropriate procedure. As mentioned above, the treatment of a cardiac valve annulus is only one example of a procedure that may benefit from the anchors described herein. In general the flexible tissue anchors described herein may be used to connect tissue to tissue or an implant or graft to a tissue, or a graft to a graft, or to form an anchoring system for reshaping tissue.

In one variation, the anchors may comprise part of an anchoring system for reshaping tissue. For example, the anchors may be implanted in tissue and cinched together using a connector (e.g., a tether or a cable) coupled thereto. The eyelet of the anchor (e.g., the loop region) may couple to a cable or tether and be cinched.

An implant or other device may be used to attach a graft or implant material to a tissue. In some variations, the anchor may pierce both the graft and the tissue, so that the anchor holds (or assists in holding) the graft to the tissue. In some variations, a cable, suture, or the like may be used to connect the anchor (e.g., through the loop region) the graft. In some variations, the anchor may connect different regions of tissue.

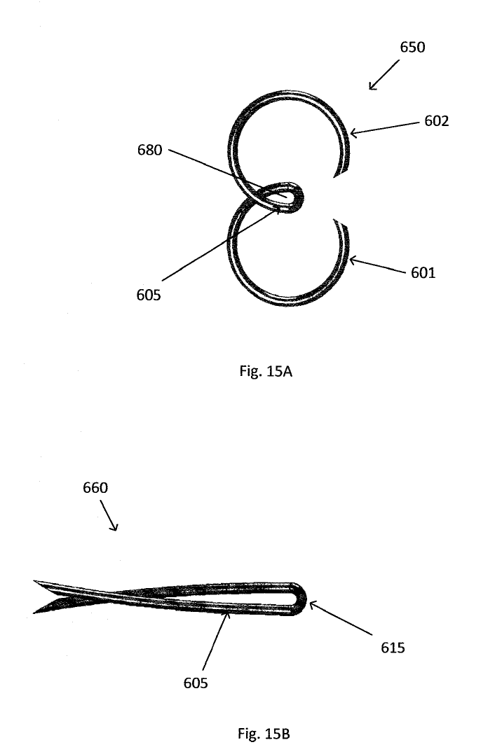

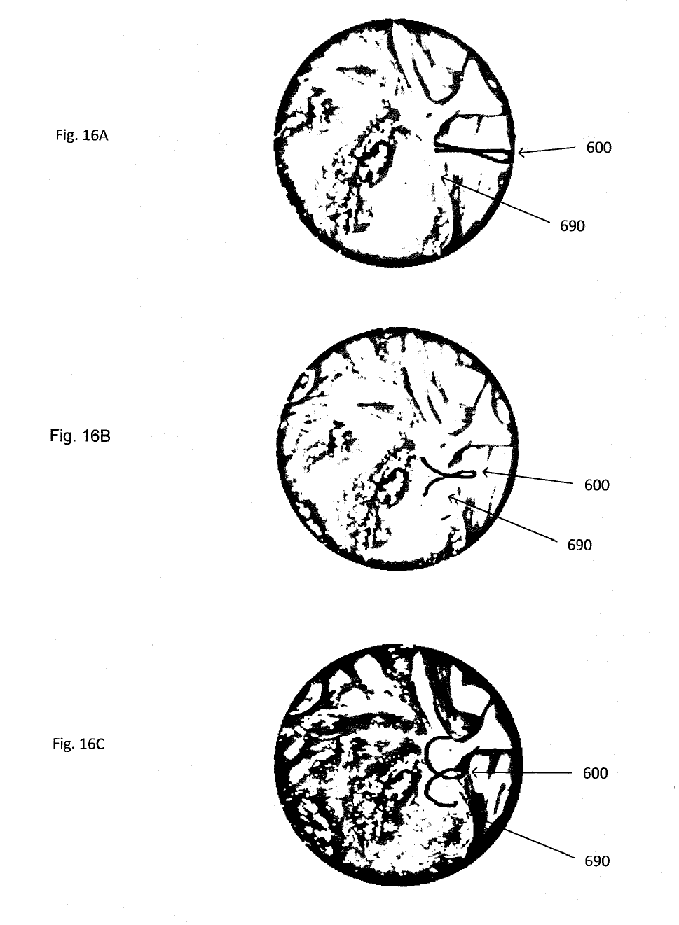

FIGS. 16A to 16C show an example of insertion of an anchor into tissue. In FIG. 16A, an anchor 600 is shown in a delivery configuration so that the legs are compressed, as described above. The legs of the anchor are shown abutting the tissue region 690 into which the anchor will be inserted. As described herein, any appropriate method of delivery of the anchor (e.g., anchor applicator, or application cannula or catheter) may be used. In FIG. 16B, the anchor is released (e.g., by an applicator) from the delivery configuration, and the legs pierce the tissue and are drawn in a curving pathway through the tissue, so that the anchor may assume the deployed configuration. As the legs are driven through the tissue in the curving pathway, the loop region becomes smaller, and the loop region of the anchor is pulled by the action of the legs into the tissue. Finally, in FIG. 16C, the anchor has expanded into the tissue and has assumed the deployed configuration in which the legs are spread out within the tissue, and the loop region is at least partly embedded in the tissue where the legs first entered the tissue.

As described above, the curved profile of the legs as they transition from a compressed to a deployed configuration result in the legs penetrating the tissue in a curved pathway. The curved pathway may further help minimize the trauma of insertion of the anchor into the tissue, and may help guide the anchor into an inserted position. In FIG. 16A-16C, the curved legs penetrate the tissue in an opposing fashion, so that deflection of the tissue by the anchor being inserted is minimized. This helps minimize compression of the tissue by the anchor ends between the legs of the anchor that might result in gathering tissue between the legs of the anchor. As the anchor expands into the deployed configuration, the leg ends curve back towards the entry site of the anchor into the tissue. As described above, this self-expanding motion may help drive the anchor into the tissue and draw the loop region into the tissue. It may be desirable to draw the loop region at least partly into the tissue to promote long-term healing and stability of the anchor within the tissue. In some variations, the anchor legs are radially extended over a broad area of the tissue when the anchor is deployed distributing forces that act on the anchor over a large area of tissue.

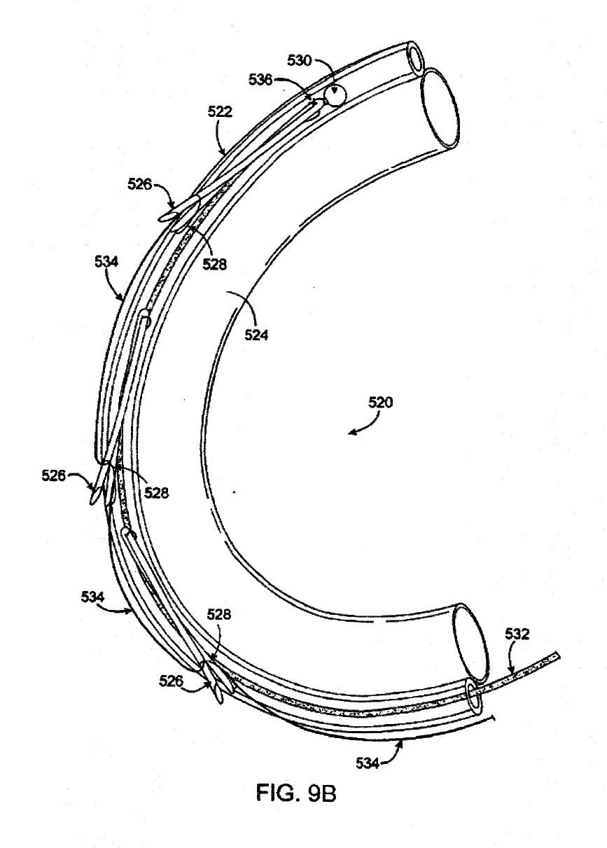

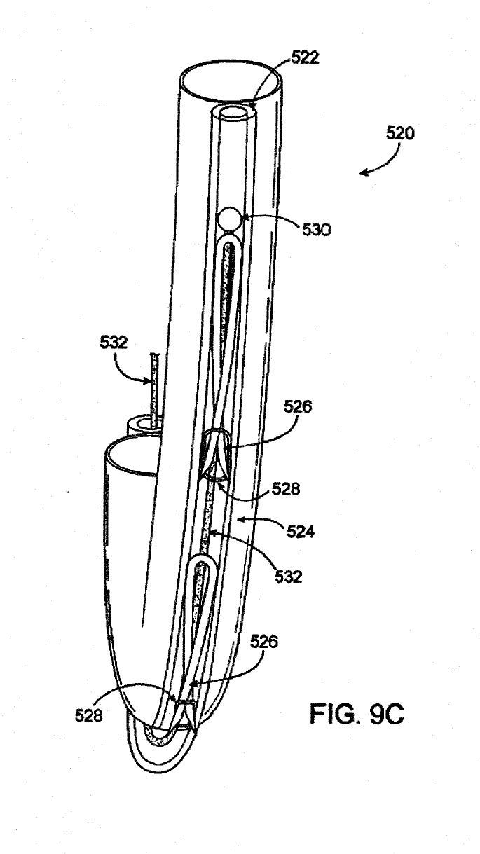

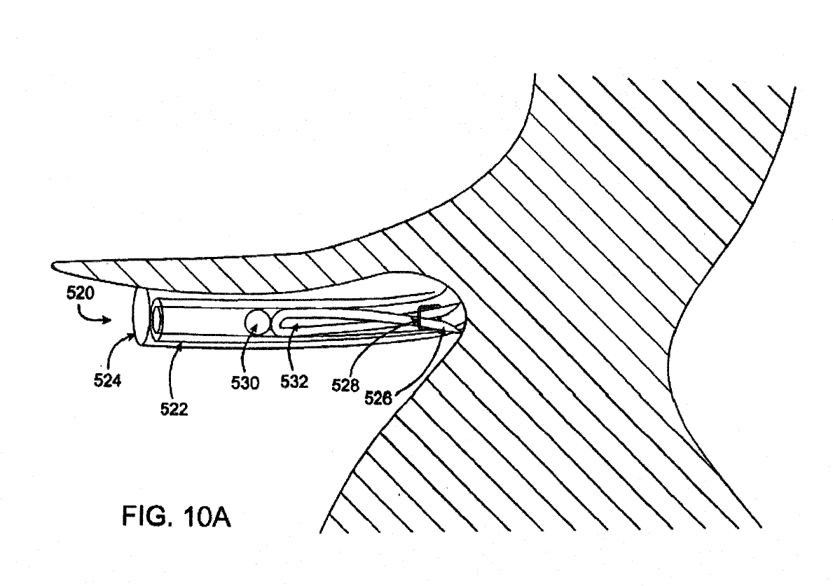

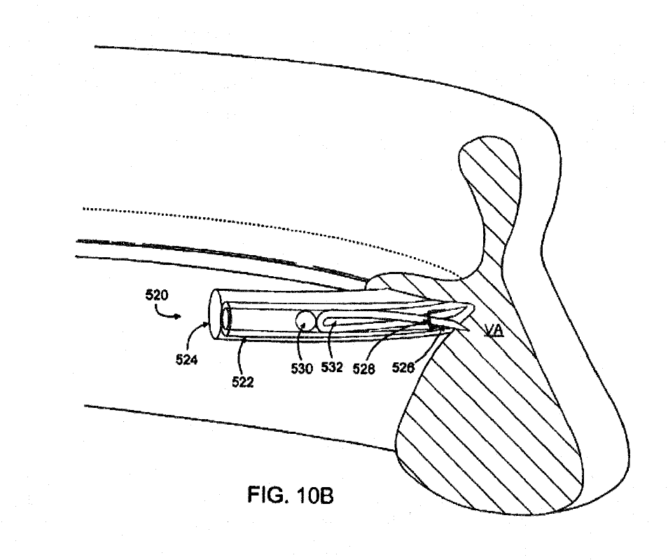

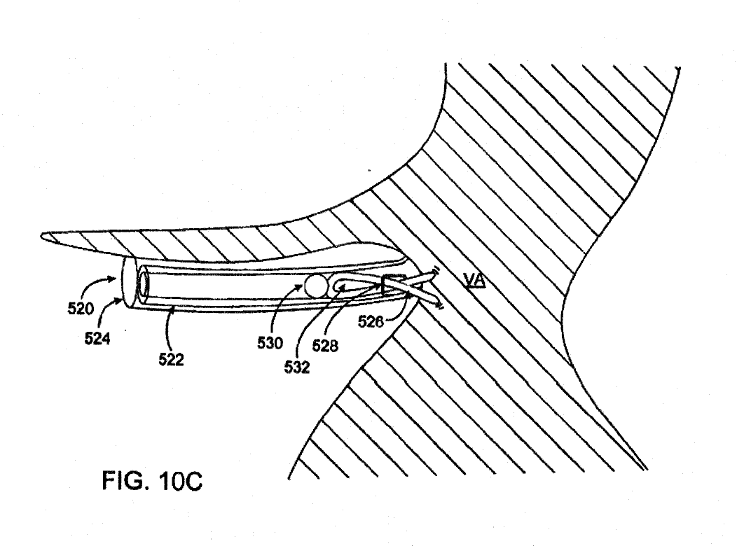

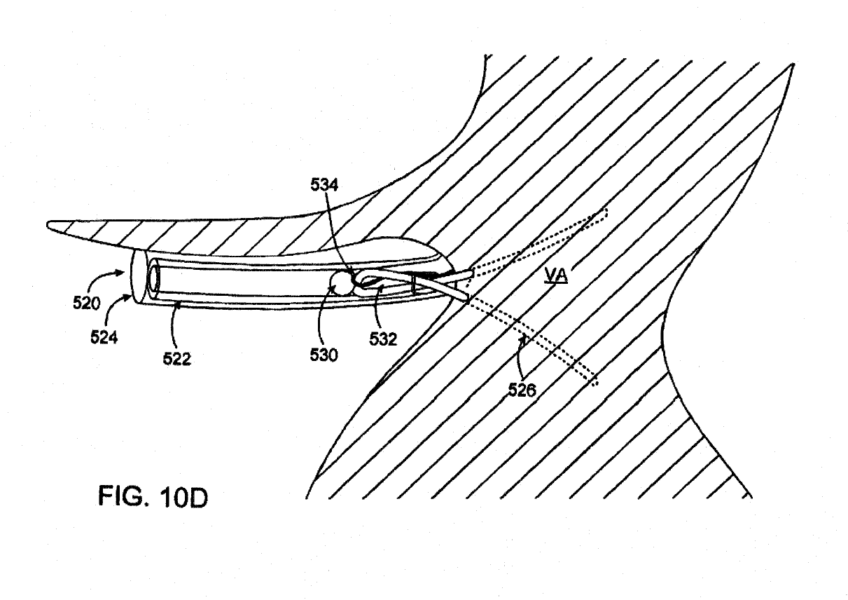

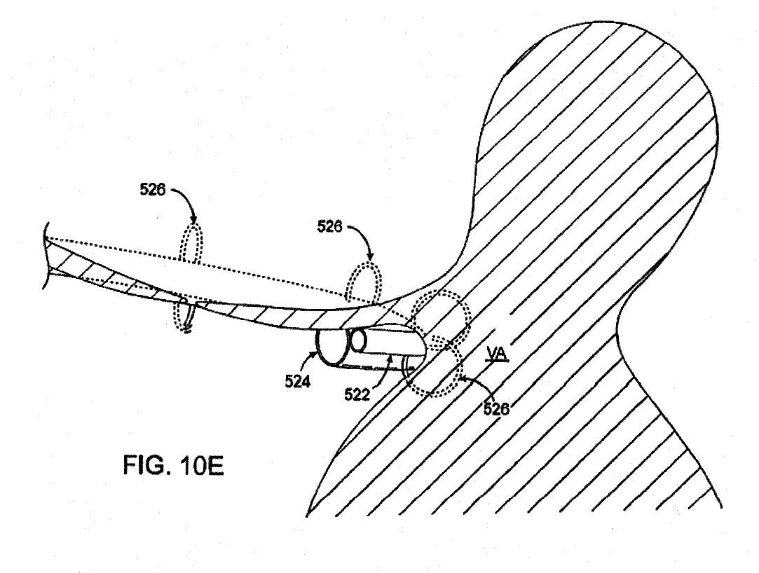

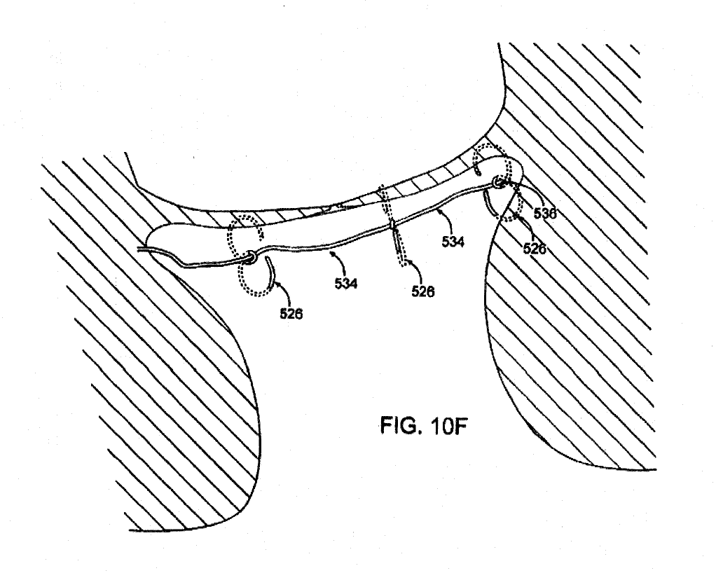

The anchor legs may be deployed in a direction that is parallel (or approximately parallel) to the direction that the anchor is inserted into the tissue or graft, as shown in FIG. 15B. In the delivery configuration, the crossover point (where the legs cross to close the loop) of the collapsed anchor is typically allowed to move or realign towards the tips of the legs. Because the anchor has a single turning direction, the crossover region of the anchor is allowed enough freedom of motion so that the legs may be oriented in parallel with the direction of deployment when the anchor is loaded in a delivery device. Thus, as shown in FIG. 15B, FIGS. 9 and 10, the ends of the legs point in approximately the same direction. Because of this leg orientation, the anchor may penetrate the tissue in the direction of deployment. In some variations, the direction of deployment is perpendicular to the surface of the tissue into which the anchor is inserted. The legs may be adapted to penetrate a tissue in a single direction, and thus, both legs may enter the tissue in the same direction. Deploying the anchors such at the legs of the anchors are substantially parallel to the direction of the deployment may allow the anchor to penetrate more deeply and more consistently than anchors whose legs deploy in an orientation that is not parallel to the direction of deployment in the delivery configuration. In particular, the ends of the legs (and a region of the leg that will enter the tissue first) should be substantially parallel to the direction of deployment. Thus, the entire length of each leg does not have to be parallel to the direction of deployment. In some variations, the legs (or the ends of the legs that may enter the tissue first) are roughly parallel to the direction of deployment. Furthermore, once the anchors are deployed, the legs may travel in a curved pathway away from the initial direction of deployment, thereby securing the anchor in the tissue.

The flexible anchors described herein may anchor within the tissue without excessively damaging (e.g., tearing, ripping or pulling out of) the tissue, because the anchor is compliant. For example, the flexible anchors described herein may flex or bend to as the tissue moves. The ability of the anchor to expand or contract in this fashion may be particularly beneficial under dynamic loading conditions. Dynamic loading conditions include repetitive or cyclic loading, such as those that might be found in muscles (e.g., heart tissue), fibrous connective tissues (e.g., tendons, ligaments), cardiovascular tissue, and other tissues. By absorbing energy that is applied during loading (e.g., repetitive loading) the anchor may lower the peak stresses on the tissue and a graft or other implant secured by the anchor. Furthermore, the elasticity of anchors applied may be matched to the elasticity of the tissue into which the anchor is inserted. Because the elasticity of the anchor is matched with the elasticity of the tissue, the anchor may expand and contract from the deployed configuration to help absorb and distribute forces acting on the anchor and the tissue in which the anchor is located.

As described herein, the anchor may be used for any appropriate procedure, including, but not limited to, annulus repair. For example, anchors may be used in place or in addition to other suturing methods, and may be useful in attaching grafts or other materials to tissue, joining tissues, or the like. The anchor may also be used as part of an anchor assembly or anchoring system. Anchors may be used for atrial septal defect closure, Gastroesophageal Reflux Disease (GERD), aneurysm repair (e.g., abdominal aortic aneurysm), ligament repair, tendon repair, repair of torn muscle, male and female urinary incontinence reduction (e.g., by reducing urethral lumen), fecal incontinence reduction, and repair of biological valves.

Another exemplary use of the anchors described herein includes using them to secure pacemaker leads. For example, the leads may be anchored by arranging the lead so that it passes though the anchor loop (eye). In some variations, the leads may by anchored using additional material, including a sheath through which the lead passes that is attached by the anchors. In some variations, the pacemaker leads are placed between the anchor legs and the tissue when the anchor is inserted.

In all of the examples described herein, these anchors may secure tissue (or secure implants, devices or grafts to the tissue) without contributing to necrosis or ischemia of the tissue. As described above, the anchors do no compress the tissue, particularly in the deployed state. Thus, the anchors may avoid tissue damage or remodeling that is associated with chronic compression of the tissue, such as tissue necrosis and ischemia.

The anchors described herein may be deployed in any appropriate tissues. As described above, anchors may transmit signals (e.g., for peacemaking) and thus may be inserted into the sinoatrial node, the atrioventricular node, Perkinjie fibers, myocardium, etc. Anchors may also be used to treat or repair patent foramen ovale (PFO), obesity (e.g., insertion into the stomach, the GI, the GI/GE junction), bowel anastamosis, appendectomy, rectal prolapse, hernia repair, uterine prolapse, bladder repair, tendon end ligament repair, joint capsulary repair, attachment of soft tissues to bone, nerve repair, etc. Anchors may also attach implants or grafts. For example, an anchor may be used to attach annuloplasty rings or valves to an annulus. The anchors described herein may also be used to close vascular access ports for percutaneous procedures.

Described below are examples and illustrations of anchors, anchor systems, and methods of using anchors.

### EXAMPLES

As mentioned above, the following examples describe the use of anchors for treating a cardiac valve annulus. These examples are only intended to illustrate one possible use of the anchors, anchor delivery devices, anchor systems, and methods of using them, and should not be considered limiting.

When used for treatment of a cardiac valve annulus, the methods described herein may involve contacting an anchor delivery device with a length of the valve annulus, delivering a plurality of coupled anchors from the anchor delivery device, and drawing the anchors together to tighten the annulus. Devices include an elongate catheter having a housing at or near the distal end for releasably housing a plurality of coupled anchors, as well as delivery devices for facilitating advancement and/or positioning of an anchor delivery device. Devices may be positioned such that the housing abuts or is close to valve annular tissue, such as in a location within the left ventricle defined by the left ventricular wall, a mitral valve leaflet and chordae tendineae. Self-securing anchors having any of a number of different configurations may be used in some variations. Additional devices include delivery devices for facilitating delivery and/or placement of an anchor delivery device at a treatment site.

In some cases, methods described herein will be performed on a beating heart. Access to the beating heart may be accomplished by any available technique, including intravascular, transthoracic, and the like. In addition to beating heart access, the methods of the described herein may be used for intravascular stopped heart access as well as stopped heart open chest procedures.

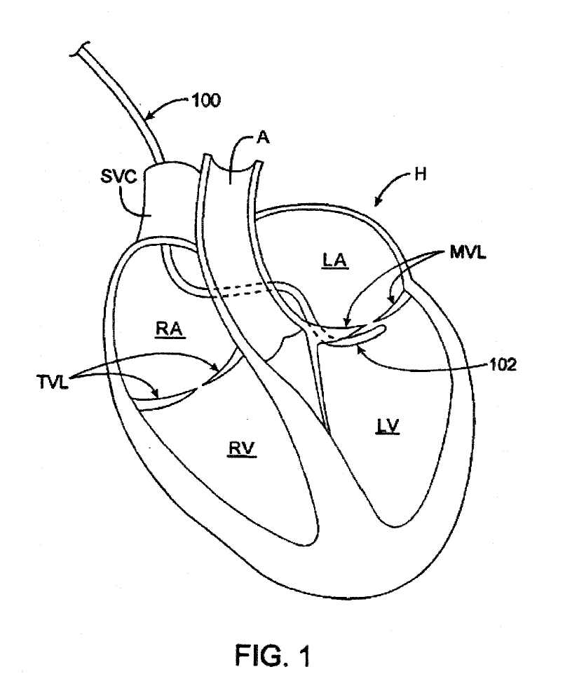

Referring now to FIG. 1, a heart H is shown in cross section, with an elongate anchor delivery device 100 introduced within the heart H. Anchors may be delivered or inserted into tissue (including heart tissue, as described below) using any appropriate delivery device. In the example shown in FIG. 1, a delivery device 100 comprises an elongate body with a distal portion 102 configured to deliver anchors to a heart valve annulus. (In FIGS. 1, 2A and 2B, distal portion 102 is shown diagrammatically without anchors or anchor-delivery mechanism to enhance clarity of the figures.) In some variations, the elongate body comprises a rigid shaft, while in other variations it comprises a flexible catheter, so that distal portion 102 may be positioned in the heart H and under one or more valve leaflets to engage a valve annulus via a transvascular approach. Transvascular access may be gained, for example, through the internal jugular vein (not shown) to the superior vena cava SVC to the right atrium RA, across the interatrial septum to the left atrium LA, and then under one or more mitral valve leaflets MVL to a position within the left ventricle (LV) under the valve annulus (not shown). Alternatively, access to the heart may be achieved via the femoral vein and the inferior vena cava. In other variations, access may be gained via the coronary sinus (not shown) and through the atrial wall into the left atrium. In still other variations, access may be achieved via a femoral artery and the aorta, into the left ventricle, and under the mitral valve. Any other suitable access route is also contemplated within the scope of the present invention.

In other variations, access to the heart H may be transthoracic, with delivery device 100 being introduced into the heart via an incision or port on the heart wall. Even open heart surgical procedures may benefit from methods and devices described herein. Furthermore, some variations may be used to enhance procedures on the tricuspid valve annulus, adjacent the tricuspid valve leaflets TVL, or any other cardiac or vascular valve. Therefore, although the following description typically focuses on minimally invasive or less invasive mitral valve repair for treating mitral regurgitation, the invention is in no way limited to that use.

With reference now to FIGS. 2A and 2B, a method for positioning delivery device 100 for treating a mitral valve annulus VA is depicted diagrammatically in a cross-sectional view. First, as in FIG. 2A, distal portion 102 is positioned in a desired location under a mitral valve leaflet L and adjacent a ventricular wall VW. (Again, distal portion 102 is shown without anchors or anchor-delivery mechanism for demonstrative purposes.) The valve annulus VA generally comprises an area of heart wall tissue at the junction of the ventricular wall VW and the atrial wall AW that is relatively fibrous and, thus, significantly stronger that leaflet tissue and other heart wall tissue.

Distal portion 102 may be advanced into position under the valve annulus by any suitable technique, some of which are described below in further detail. Generally, distal portion 102 may be used to deliver anchors to the valve annulus, to stabilize and/or expose the annulus, or both. In one variation, using a delivery device having a flexible elongate body as shown in FIG. 1, a flexible distal portion 102 may be passed from the right atrium RA through the interatrial septum in the area of the foramen ovale (not shown—behind the aorta A), into the left atrium LA and thus the left ventricle LV. Alternatively, flexible distal portion 102 may be advanced through the aorta A and into the left ventricle LV, for example using access through a femoral artery. Oftentimes, distal portion 102 will then naturally travel, upon further advancement, under the posterior valve leaflet L into a space defined above a subvalvular space 104 roughly defined for the purposes of this application as a space bordered by the inner surface of the left ventricular wall VW, the inferior surface of mitral valve leaflets L, and cordae tendineae CT connected to the ventricular wall VW and the leaflet L. It has been found that a flexible anchor delivery catheter, such as the delivery devices described herein, when passed under the mitral valve via an intravascular approach, often enters subvalvular space 104 relatively easily and may be advanced along space 104 either partially or completely around the circumference of the valve. Once in space 104, distal portion 102 may be conveniently positioned at the intersection of the valve leaflet(s) and the ventricular wall VW, which intersection is immediately adjacent or very near to the valve annulus VA, as shown in FIG. 2A. These are but examples of possible access routes of an anchor delivery device to a valve annulus, and any other access routes may be used.

In some variations, distal portion 102 includes a shape-changing portion which enables distal portion 102 to conform to the shape of the valve annulus VA. The catheter may be introduced through the vasculature with the shape-changing distal portion in a generally straight, flexible configuration. Once it is in place beneath the leaflet at the intersection between the leaflet and the interior ventricular wall, the shape of distal portion 102 is changed to conform to the annulus and usually the shape is “locked” to provide sufficient stiffness or rigidity to permit the application of force from distal portion 102 to the annulus. Shaping and optionally locking distal portion 102 may be accomplished in any of a number of ways. For example, in some variations, a shape-changing portion may be sectioned, notched, slotted or segmented and one of more tensioning members such as tensioning cords, wires or other tensioning devices coupled with the shape-changing portion may be used to shape and rigidify distal portion 102. A segmented distal portion, for example, may include multiple segments coupled with two tensioning members, each providing a different direction of articulation to the distal portion. A first bend may be created by tensioning a first member to give the distal portion a C-shape or similar shape to conform to the valve annulus, while a second bend may be created by tensioning a second member to articulate the C-shaped member upwards against the annulus. In another variation, a shaped expandable member, such as a balloon, may be coupled with distal portion 102 to provide for shape changing/deforming. In various variations, any configurations and combinations may be used to give distal portion 102 a desired shape.

In transthoracic and other variations, distal portion 102 may be pre-shaped, and the method may simply involve introducing distal portion 102 under the valve leaflets. The pre-shaped distal portion 102 may be rigid or formed from any suitable super-elastic or shape memory material, such as Nitinol, spring stainless steel, or the like.

In addition to delivering anchors to the valve annulus VA, delivery device 100 (and specifically distal portion 102) may be used to stabilize and/or expose the valve annulus VA. Such stabilization and exposure are described fully in U.S. patent application Ser. No. 10/656,797, which was previously incorporated by reference. For example, once distal portion 102 is positioned under the annulus, force may be applied to distal portion 102 to stabilize the valve annulus VA, as shown in FIG. 2B. Such force may be directed in any suitable direction to expose, position and/or stabilize the annulus. For example, upward and lateral force is shown in FIG. 2B by the solid-headed arrow drawn from the center of distal portion 102. In other cases, only upward, only lateral, or any other suitable force(s) may be applied. With application of force to distal portion 102, the valve annulus VA is caused to rise or project outwardly, thus exposing the annulus for easier viewing and access. The applied force may also stabilize the valve annulus VA, also facilitating surgical procedures and visualization.

Some variations may include a stabilization component as well as an anchor delivery component. For example, some variations may include two flexible members, one for contacting the atrial side of a valve annulus and the other for contacting the ventricular side. In some variations, such flexible members may be used to “clamp” the annulus between them. One of such members may be an anchor delivery member and the other may be a stabilization member, for example. Any combination and configuration of stabilization and/or anchor delivery members is contemplated.

Referring now to FIGS. 2C and 2D, an anchor delivery device 108 is shown delivering an anchor 110 to a valve annulus VA. Of course, these are again representational figures and are not drawn to scale. One variation of an anchor 110 is shown first housed within delivery device 108 (FIG. 2C) and then delivered to the annulus VA (FIG. 2D). As is shown, in one variation anchors 110 may have a relatively straight configuration when housed in delivery device 108, perhaps with two sharpened tips and a loop in between the tips. Upon deployment from delivery device 108, the tips of anchor 110 may curve in opposite directions to form two semi-circles, circles, ovals, overlapping helices or the like. This is but one example of a type of self-securing anchor which may be delivered to a valve annulus. Typically, multiple coupled anchors 110 are delivered, and the anchors 110 are drawn together to tighten the valve annulus. Methods for anchor delivery and for drawing anchors together are described further below.

Although delivery device 108 is shown having a circular cross-sectional shape in FIGS. 2C and 2D, it may alternatively have any other suitable shape. In one variation, for example, it may be advantageous to provide a delivery device having an ovoid or elliptical cross-sectional shape. Such a shape may help ensure that the device is aligned, when positioned between in a corner formed by a ventricular wall and a valve leaflet, such that one or more openings in the delivery device is oriented to deliver the anchors into valve annulus tissue. To further enhance contacting of the valve annulus and/or orientation of the delivery device, some variations may further include an expandable member, coupled with the delivery device, which expands to urge or press or wedge the delivery device into the corner formed by the ventricle wall and the leaflet to contact the valve annulus. Such enhancements are described further below.

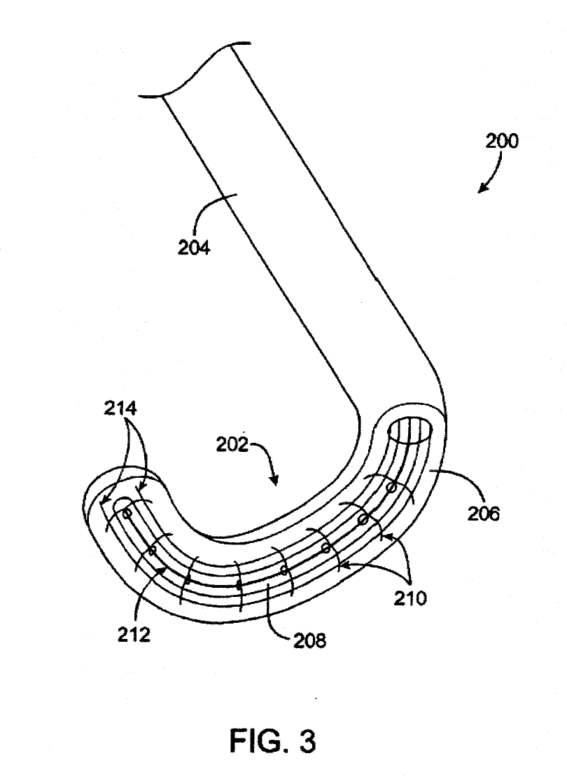

With reference now to FIG. 3, one variation of a portion of an anchor delivery device 200 suitably includes an elongate shaft 204 having a distal portion 202 configured to deliver a plurality of anchors 210, coupled with a tether 212, to tissue of a valve annulus. Tethered anchors 210 are housed within a housing 206 of distal portion 202, along with one or more anchor retaining mandrels 214 and an expandable member 208. Many variations may be made to one or more of these features, and various parts may be added or eliminated, without departing from the scope of the invention. Some of these variations are described further below, but no specific variation(s) should be construed to limit the scope of the invention as defined by the appended claims.

Housing 206 may be flexible or rigid in various variations. In some variations, for example, flexible housing 206 may be comprised of multiple segments configured such that housing 206 is deformable by tensioning a tensioning member coupled to the segments. In some variations, housing 206 is formed from an elastic material having a geometry selected to engage and optionally shape or constrict the valve annulus. For example, the rings may be formed from super-elastic material, shape memory alloy such as Nitinol, spring stainless steel, or the like. In other instances, housing 206 could be formed from an inflatable or other structure can be selectively rigidified in situ, such as a gooseneck or lockable element shaft, any of the rigidifying structures described above, or any other rigidifying structure.

As described above, in some variations, anchors 210 may comprise C-shaped or semicircular hooks, curved hooks of other shapes, straight hooks, barbed hooks, clips of any kind, T-tags, or any other suitable fastener(s). In one variation, as described above, anchors may comprise two tips that curve in opposite directions upon deployment, forming two intersecting semi-circles, circles, ovals, helices or the like. In some variations, anchors 210 are self-deforming. By “self-deforming” it is meant that anchors 210 change from a first undeployed shape to a second deployed shape upon release of anchors 210 from restraint in housing 206. Such self-deforming anchors 210 may change shape as they are released from housing 206 and enter valve annulus tissue, to secure themselves to the tissue. Thus, a crimping device or other similar mechanism is not required on distal end 202 to apply force to anchors 210 to attach them to annular tissue. Self-deforming anchors 210 may be made of any suitable material, such as a super-elastic or shape-memory material like Nitinol or spring stainless steel. In other variations, anchors 210 may be made of a non-shape-memory material and made be loaded into housing 206 in such a way that they change shape upon release. Alternatively, anchors 210 that are not self-deforming may be used, and such anchors may be secured to tissue via crimping, firing or the like. Even self-securing anchors may be crimped in some variations, to provide enhanced attachment to tissue. Delivery of anchors may be accomplished by any suitable device and technique, such as by simply releasing the anchors by hydraulic balloon delivery as discussed further below. Any number, size and shape of anchors 210 may be included in housing 206.

In one variation, anchors 210 are generally C-shaped or semicircular in their undeployed form, with the ends of the C being sharpened to penetrate tissue. Midway along the C-shaped anchor 210, an eyelet may be formed for allowing slidable passage of tether 212. To maintain anchors 210 in their C-shaped, undeployed state, anchors 210 may be retained within housing 206 by two mandrels 214, one mandrel 214 retaining each of the two arms of the C-shape of each anchor 210. Mandrels 214 may be retractable within elongate catheter body 204 to release anchors 210 and allow them to change from their undeployed C-shape to a deployed shape. The deployed shape, for example, may approximate a complete circle or a circle with overlapping ends, the latter appearing similar to a key ring. Such anchors are described further below, but generally may be advantageous in their ability to secure themselves to annular tissue by changing from their undeployed to their deployed shape. In some variations, anchors 210 are also configured to lie flush with a tissue surface after being deployed. By “flush” it is meant that no significant amount of an anchor protrudes from the surface, although some small portion may protrude.

Tether 212 may be one long piece of material or two or more pieces and may comprise any suitable material, such as suture, suture-like material, a Dacron strip or the like. Retaining mandrels 214 may also have any suitable configuration and be made of any suitable material, such as stainless steel, titanium, Nitinol, or the like. Various variations may have one mandrel, two mandrels, or more than two mandrels.

In some variations, anchors 210 may be released from mandrels 214 to contact and secure themselves to annular tissue without any further force applied by delivery device 200. Some variations, however, may also include one or more expandable members 208, which may be expanded to help drive anchors 210 into tissue. Expandable member(s) 208 may have any suitable size and configuration and may be made of any suitable material(s). Hydraulic systems such as expandable members are known in the art, and any known or as yet undiscovered expandable member may be included in housing 206.

Referring now to FIGS. 4 and 5, a segment of a distal portion 302 of an anchor delivery device suitably includes a housing 306, multiple tensioning members 320 for applying tension to housing 306 to change its shape, two anchor retaining mandrels 314 slideably disposed in housing 306, multiple anchors 310 slideably coupled with a tether 312, and an expandable member 308 disposed between anchors 310 and housing 306. As can be seen in FIGS. 4 and 5, housing 306 may include multiple segments to allow the overall shape of housing 306 to be changed by applying tension to tensioning members 320. As is also evident from the drawings, anchors 310 may actually have an almost straight configuration when retained by mandrels 314 in housing 306 an may be “C-shaped” when deployed. “C-shaped” or “semicircular” may refer to a very broad range of shapes including a portion of a circle, a slightly curved line, a slightly curved line with an eyelet at one point along the line, and the like.

With reference now to FIG. 6, the same segment of distal portion 302 is shown, but mandrels 314 have been withdrawn from two mandrel apertures 322, to release anchors 310 from housing 306. Additionally, expandable member 308 has been expanded to drive anchors out of housing 306. Anchors 310, having been released from mandrels 314, have begun to change from their undeployed, retained shape to their deployed, released shape.

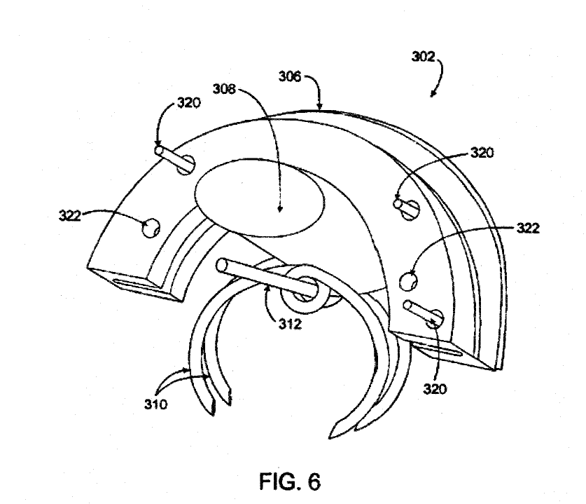

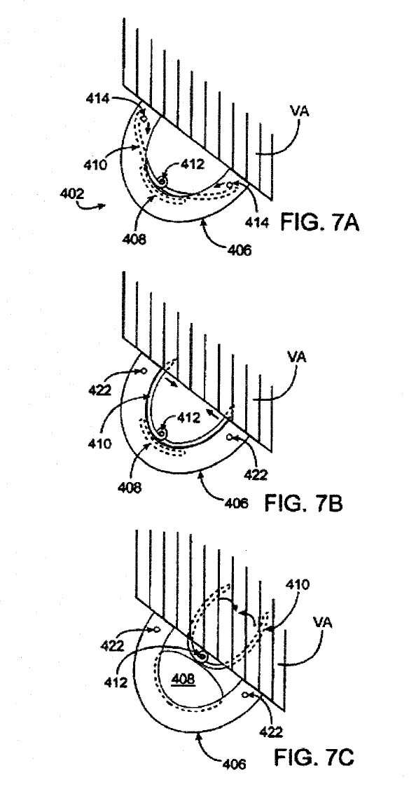

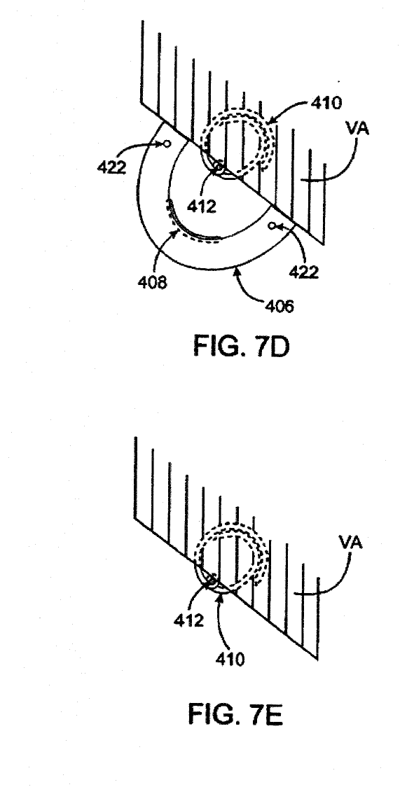

Referring now to FIGS. 7A-7E, a cross-section of a distal portion 402 of an anchor delivery device is shown in various stages of delivering an anchor to tissue of a valve annulus VA. In FIG. 7A, distal portion 402 is positioned against the valve annulus, an anchor 410 is retained by two mandrels 414, a tether 412 is slideably disposed through an eyelet on anchor 410, and an expandable member 408 is coupled with housing 406 in a position to drive anchor 410 out of housing 406. When retained by mandrels 414, anchor 410 is in its undeployed shape. As discussed above, mandrels 414 may be slideably retracted, as designated by the solid-tipped arrows in FIG. 7A, to release anchor 410. In various variations, anchors 410 may be released one at a time, such as by retracting mandrels 414 slowly, may be released in groups, or may all be released simultaneously, such as by rapid retraction of mandrels 414.

In FIG. 7B, anchor 410 has begun to change from its undeployed shape to its deployed shape (as demonstrated by the hollow-tipped arrows) and has also begun to penetrate the annular tissue VA. Empty mandrel apertures 422 demonstrate that mandrels 414 have been retracted at least far enough to release anchor 410. In FIG. 7B, expandable member 408 has been expanded to drive anchor 410 partially out of housing 406 and further into the valve annulus VA. Anchor 410 also continues to move from its undeployed towards its deployed shape, as shown by the hollow-tipped arrows. In FIG. 7D, anchor 410 has reached its deployed shape, which is roughly a completed circle with overlapping ends or a “key ring” shape. In FIG. 7E, delivery device 402 has been removed, leaving a tethered anchor in place in the valve annulus. Of course, there will typically be a plurality of tethered anchors secured to the annular tissue. Tether 412 may then be cinched to apply force to anchors 410 and cinch and tighten the valve annulus.

The anchors described in FIG. 7 comprise a variation having a deployed configuration that is a loop or semicircle. As previously described, in some variations the legs (e.g., the tips of the legs) are extended in the deployed configuration so that the anchor has the greatest “span” in the deployed configuration. For example, the deployed configuration may resemble the undeployed or delivery configuration described above in FIG. 7A.

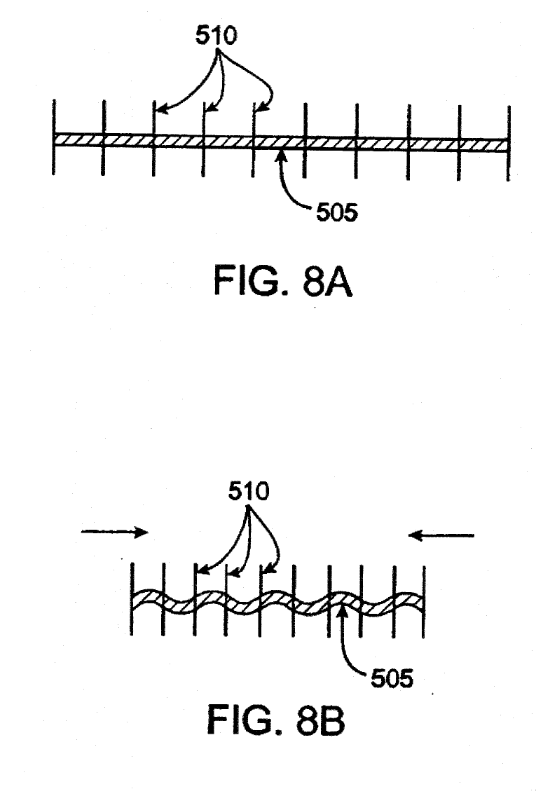

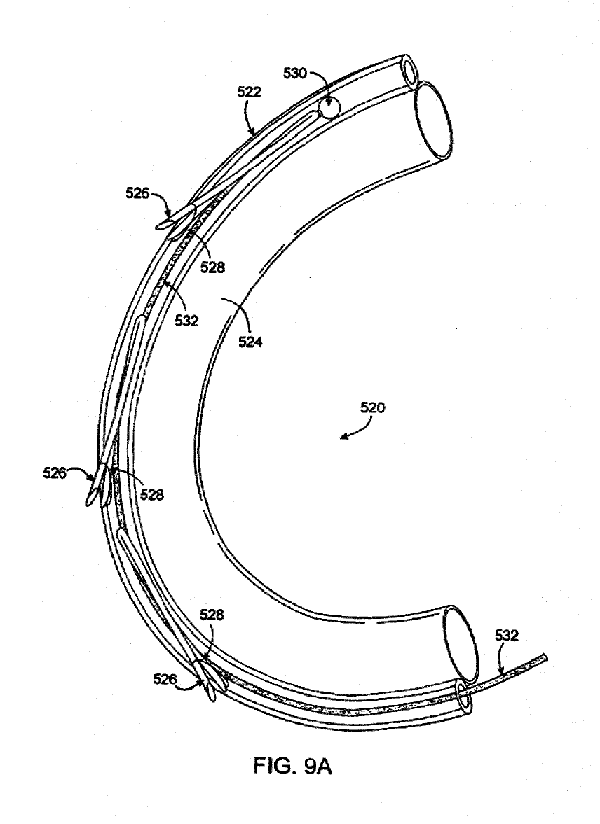

With reference now to FIGS. 8A and 8B, a diagrammatic representation of another variation of coupled anchors is shown. Here, anchors 510 are coupled to a self-deforming or deformable coupling member or backbone 505. Backbone 505 may be fabricated, for example, from Nitinol, spring stainless steel, or the like, and may have any suitable size or configuration. In one variation, as in FIG. 8A, backbone 505 is shaped as a generally straight line when held in an undeployed state, such as when restrained within a housing of an anchor deliver device. When released from the delivery device, backbone 505 may change to a deployed shape having multiple bends, as shown in FIG. 8B. By bending, backbone 505 shortens the longitudinal distance between anchors, as demonstrated by the solid-tipped arrows in FIG. 8B. This shortening process may act to cinch a valve annulus into which anchors 510 have be secured. Thus, anchors 510 coupled to backbone 505 may be used to cinch a valve annulus without using a tether or applying tethering force. Alternatively, a tether may also be coupled with anchors 510 to further cinch the annulus. In such a variation, backbone 505 will be at least partially conformable or cinchable, such that when force is applied to anchors 510 and backbone 505 via a tether, backbone 505 bends further to allow further cinching of the annulus.

Referring now to FIGS. 9A-9C, in one variation a flexible distal portion of an anchor delivery device 520 suitably includes a housing 522 coupled with an expandable member 524. Housing 522 may be configured to house multiple coupled anchors 526 and an anchor contacting member 530 coupled with a pull cord 532. Housing 522 may also include multiple apertures 528 for allowing egress of anchors 526. For clarity, delivery device 520 is shown without a tether in FIGS. 9A and 9C, but FIG. 9B shows that a tether 534 may extend through an eyelet, loop or other portion of each anchor 526, and may exit each aperture 528 to allow for release of the plurality of anchors 526. The various features of this variation are described further below.

In the variation shown in FIGS. 9A-9C, anchors 526 are relatively straight and lie relatively in parallel with the long axis of delivery device 522. Anchor contacting member 530, which may comprise any suitable device, such as a ball, plate, hook, knot, plunger, piston, or the like, generally has an outer diameter that is nearly equal to or slightly less than the inner diameter of housing 522. Contacting member 530 is disposed within the housing, distal to a distal-most anchor 526, and is retracted relative to housing 522 by pulling pull cord 532. When retracted, anchor contacting member 530 contacts and applies force to a distal-most anchor 526 to release cause that anchor 526 to exit housing 522 via one of the apertures 528. Contacting member 530 is then pulled farther proximally to contact and apply force to the next anchor 526 to deploy that anchor 526, and so on.

Retracting contacting member 530 to push anchors 526 out of apertures 528 may help cause anchors 526 to avidly secure themselves to adjacent tissue. Using anchors 526 that are relatively straight/flat when undeployed allows anchors 526 with relatively large deployed sizes to be disposed in (and delivered from) a relatively small housing 522. In one variation, for example, anchors 526 that deploy into a shape approximating two intersecting semi-circles, circles, ovals, helices, or the like, and that have a radius of one of the semi-circles of about 3 mm may be disposed within a housing 522 having a diameter of about 5 French (1.67 mm) and more preferably 4 French (1.35 mm) or even smaller. Such anchors 526 may measure about 6 mm or more in their widest dimension. These are only examples, however, and other larger or smaller anchors 526 may be disposed within a larger or smaller housing 522. Furthermore, any convenient number of anchors 526 may be disposed within housing 522. In one variation, for example, housing 522 may hold about 1-20 anchors 526, and more preferably about 3-10 anchors 526. Other variations may hold more anchors 526.

Anchor contacting member 530 and pull cord 532 may have any suitable configuration and may be manufactured from any material or combination of materials. In alternative variations, contacting member 530 may be pushed by a pusher member to contact and deploy anchors 526. Alternatively, any of the anchor deployment devices and methods previously described may be used.

Tether 534, as shown in FIG. 9B, may comprise any of the tethers 534 or tether-like devices already described above, or any other suitable device. Tether 534 is generally attached to a distal-most anchor 526 at an attachment point 536. The attachment itself may be achieved via a knot, weld, adhesive, or by any other suitable attachment means. Tether 234 then extends through an eyelet, loop or other similar configuration on each on each of the anchors 526 so as to be slideably coupled with the anchors 526. In the variation shown, tether 534 exits each aperture 528, then enters the next-most-proximal aperture, passes slideably through a loop on an anchor 526, and exits the same aperture 528. By entering and exiting each aperture 528, tether 534 allows the plurality of anchors 526 to be deployed into tissue and cinched. Other configurations of housing 522, anchors 526 and tether 534 may alternatively be used. For example, housing 522 may include a longitudinal slit through which tether 534 may pass, thus allowing tether 534 to reside wholly within housing before deployment.

Expandable member 524 is an optional feature of anchor delivery device 520, and thus may be included in some variations and not in others. In other words, a distal portion of anchor delivery device 520 may include housing, contents of housing, and other features either with or without an attached expandable member. Expandable member 524 may comprise any suitable expandable member currently known or discovered in the future, and any method and substance(s) may be used to expand expandable member 524. Typically, expandable member 524 will be coupled with a surface of housing 522, will have a larger radius than housing 522, and will be configured such that when it is expanded as housing 522 nears or contacts the valve annulus, expandable member 524 will push or press housing 522 into enhanced contact with the annulus. For example, expandable member 524 may be configured to expand within a space near the corner formed by a left ventricular wall and a mitral valve leaflet.

With reference now to FIGS. 10A-10F, a method is shown for applying a plurality of tethered anchors 526 to a valve annulus VA in a heart. As shown in FIG. 10A, an anchor delivery device 520 is first contacted with the valve annulus VA such that openings 528 are oriented to deploy anchors 526 into the annulus. Such orientation may be achieved by any suitable technique. In one variation, for example, a housing 522 having an elliptical cross-sectional shape may be used to orient openings 528. As just described, contact between housing 522 and the valve annulus VA may be enhanced by expanding expandable member 524 to wedge housing within a corner adjacent the annulus.

Generally, delivery device 520 may be advanced into any suitable location for treating any valve by any suitable advancing or device placement method. Many catheter-based, minimally invasive devices and methods for performing intravascular procedures, for example, are well known, and any such devices and methods, as well as any other devices or method later developed, may be used to advance or position delivery device 520 in a desired location. For example, in one variation a steerable guide catheter is first advanced in retrograde fashion through an aorta, typically via access from a femoral artery. The steerable catheter is passed into the left ventricle of the heart and thus into the space formed by the mitral valve leaflets, the left ventricular wall and cordae tendineae of the left ventricle. Once in this space, the steerable catheter is easily advanced along a portion (or all) of the circumference of the mitral valve. A sheath is advanced over the steerable catheter within the space below the valve leaflets, and the steerable catheter is removed through the sheath. Anchor delivery device 520 may then be advanced through the sheath to a desired position within the space, and the sheath may be removed. In some cases, an expandable member coupled to delivery device 520 may be expanded to wedge or otherwise move delivery device 520 into the corner formed by the left ventricular wall and the valve leaflets to enhance its contact with the valve annulus. Of course, this is but one exemplary method for advancing delivery device 520 to a position (e.g., for treating a valve), and any other suitable method, combination of devices, etc. may be used.

As shown in FIG. 10B, when delivery device 520 is positioned in a desired location for deploying anchors 526, anchor contacting member 530 is retracted to contact and apply force to a most-distal anchor 526 to begin deploying anchor 526 through aperture 528 and into tissue of the valve annulus VA. FIG. 10C show anchor 526 further deployed out of aperture 528 and into valve annulus VA. FIG. 10D shows the valve annulus VA transparently so that further deployment of anchors 526 can be seen. As shown, in one variation, anchors 526 include two sharpened tips that move in opposite directions upon release from housing 522 and upon contacting the valve annulus VA. Between the two sharpened tips, an anchor 526 may be looped or have any other suitable eyelet or other device for allowing slidable coupling with a tether 534.

Referring now to FIG. 10E, one variation of the anchors 526 are seen in a fully deployed or nearly fully deployed shape, with each pointed tip (or “arm”) of each anchor 526 having curved to form a circle or semi-circle. Of course, in various variations, anchors 526 may have any other suitable deployed and undeployed shapes, as described more fully above. FIG. 10F shows anchors 526 deployed into the valve annulus VA and coupled with tether 534, with the distal-most anchor 526 coupled attached fixedly to tether 524 at attachment point 536. At this stage, tether 534 may be cinched to tighten the annulus, thus reducing valve regurgitation. In some variations, valve function may be monitored by means such as echocardiogram and/or fluoroscopy, and tether 534 may be cinched, loosened, and adjusted to achieve a desired amount of tightening as evident via the employed visualization technique(s). When a desired amount of tightening is achieved, tether 534 is then attached to a most-proximal anchor 526 (or two or more most-proximal anchors 526), using any suitable technique, and tether 534 is then cut proximal to the most-proximal anchor 526, thus leaving the cinched, tethered anchors 526 in place along the valve annulus VA. Attachment of tether 534 to the most-proximal anchor(s) 526 may be achieved via adhesive, knotting, crimping, tying or any other technique, and cutting tether 534 may also be performed via any technique, such as with a cutting member coupled with housing 522.

In one variation, cinching tether 534, attaching tether 534 to most-proximal anchor 526, and cutting tether 534 are achieved using a termination device (not shown). The termination device may comprise, for example, a catheter advanceable over tether 534 that includes a cutting member and a Nitinol knot or other attachment member for attaching tether 534 to most-proximal anchor. The termination catheter may be advanced over tether 534 to a location at or near the proximal end of the tethered anchors 526. It may then be used to apply opposing force to the most-proximal anchor 526 while tether 534 is cinched. Attachment and cutting members may then be used to attach tether 534 to most-proximal anchor 526 and cut tether 534 just proximal to most-proximal anchor 526. Such a termination device is only one possible way of accomplishing the cinching, attachment and cutting steps, and any other suitable device(s) or technique(s) may be used.

In some variations, it may be advantageous to deploy a first number of anchors 526 along a first portion of a valve annulus VA, cinch the first anchors to tighten that portion of the annulus, move the delivery device 520 to another portion of the annulus, and deploy and cinch a second number of anchors 526 along a second portion of the annulus. Such a method may be more convenient, in some cases, than extending delivery device 520 around all or most of the circumference of the annulus, and may allow a shorter, more maneuverable housing 522 to be used.

Referring now to FIG. 11, a cross-sectional depiction of a heart H is shown with an anchor delivery device guide catheter 550 advanced through the aorta A and into the left ventricle LV. Guide catheter 550 is generally a flexible elongate catheter which may have one or more curves or bends toward its distal end to facilitate placement of the distal end of catheter 550 in a subannular space 552. Subannular space 552, which has been described above in detail, is generally defined by the left ventricular wall, the mitral valve leaflets MVL, and cordae tendiniae, and travels along most or all of the circumference of the valve annulus. The distal end of guide catheter 550 may be configured to be positioned at an opening into space 552 or within space 552, such that subsequent catheter devices may be passed through guide catheter 550 into space 552.

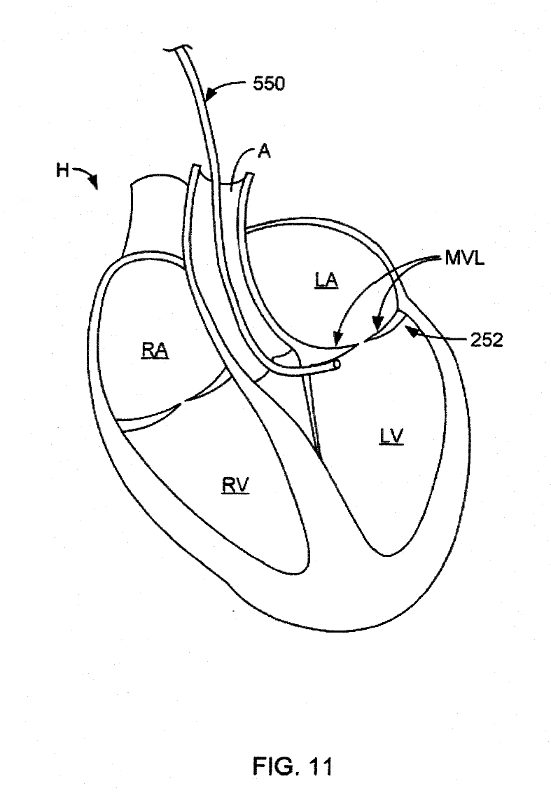

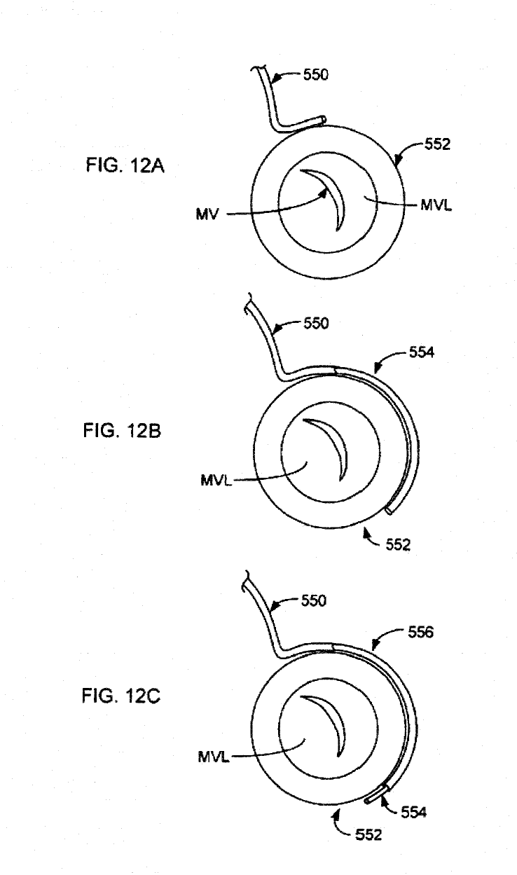

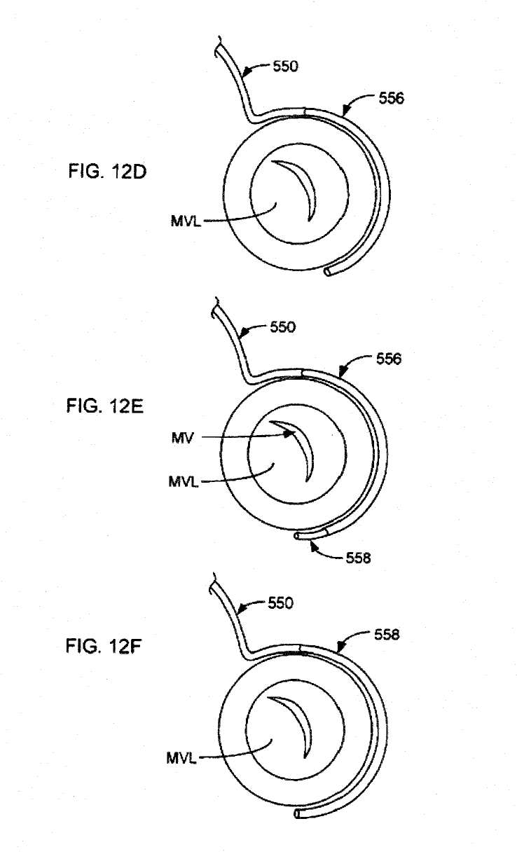

This can be more easily understood with reference to FIGS. 12A-12F, which demonstrate a method for advancing an anchor delivery device to a position for treating a mitral valve MV. The mitral valve MV, including mitral valve leaflets MVL are represented diagrammatically from an inferior perspective looking up, to depict a method for delivering a device into subannular space 552. In FIG. 12A, first guide catheter 550 is show extending up to or into subannular space 552, as in FIG. 11. As shown in FIG. 12B, in one method a second guide catheter 554 may be advanced through first guide catheter 550 to pass through/along subannular space 554. This second guide catheter 554 is steerable in one variation, as will be described further below, to help conform second guide catheter 554 to subannular space 552.

Next, as in FIG. 12C, a guide sheath 556 may be passed over second guide catheter 554 to extend along subannular space. Sheath 556 is generally a flexible, tubular member that can be passed over second guide catheter 554 and within first guide catheter 550. To enhance passage and exchange, any of these and other described catheter members, sheath members, or the like may be manufactured from and/or coated with one or more friction resistant materials. Once sheath 556 is in place, second guide catheter 554 may be withdrawn, as shown in FIG. 12D. As shown in FIG. 12E, an anchor delivery device 558 may then be advanced through sheath 556 to a position for treating the mitral valve MV. Sheath 556 may then be withdrawn, as in FIG. 12F, leaving anchor delivery device 558 in place for performing a treatment. A valve annulus treatment may be performed, as described extensively above, and anchor delivery device 558 may be withdrawn. In some variations, anchor delivery device 558 is used to treat one portion of the valve annulus and is then moved to another portion, typically the opposite side, to treat the other portion of the annulus. In such variations, any one or more of the steps just described may be repeated. In some variations, anchor delivery device 558 is withdrawn through first guide catheter 550, and first guide catheter 550 is then withdrawn. In alternative variations, first guide catheter 550 may be withdrawn before anchor delivery device 558.

In various variations, alternative means may be used to urge anchor delivery device 558 into contact with the valve annulus. For example, in one variation an expandable member is coupled with anchor delivery device 558 and expanded within the subannular space 552. In an alternative variation, a magnet may be coupled with anchor delivery device 558, and another anchor may be disposed within the coronary sinus, in proximity to the first magnet. The two magnets may attract one another, thus pulling the anchor delivery device 558 into greater contact with the annulus. These or other variations may also include visualizing the annulus using a visualization member coupled with the anchor delivery device 558 or separate from the device 558. In some variations, anchors may be driven through a strip of detachable, biocompatible material, such as Dacron, that is coupled with anchor delivery device 558 but that detaches to affix to the valve annulus via the anchors. In some variations, the strip may then be cinched to tighten the annulus. In other variations, the anchors may be driven through a detachable, biocompatible, distal portion of the guide sheath 556, and guide sheath 556 may then remain attached to the annulus via the anchors. Again, in some variations, the detached sheath may be cinched to tighten the annulus.

Of course, the method just described is but one variation of a method for delivering an anchor delivery device to a location for treating a valve annulus. In various alternative variations, one or more steps may be added, deleted or modified while achieving a similar result. In some variations, a similar method may be used to treat the mitral valve from a superior/right atrial position or to treat another heart valve. Additionally, other devices or modifications of the system just described may be used in other variations.

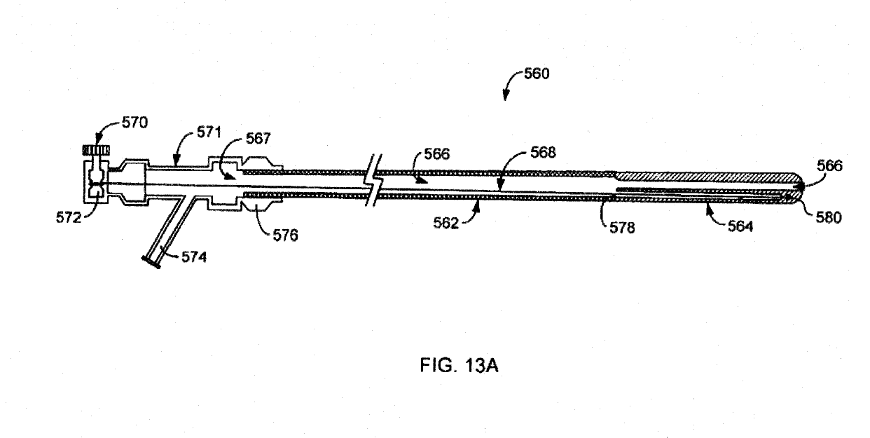

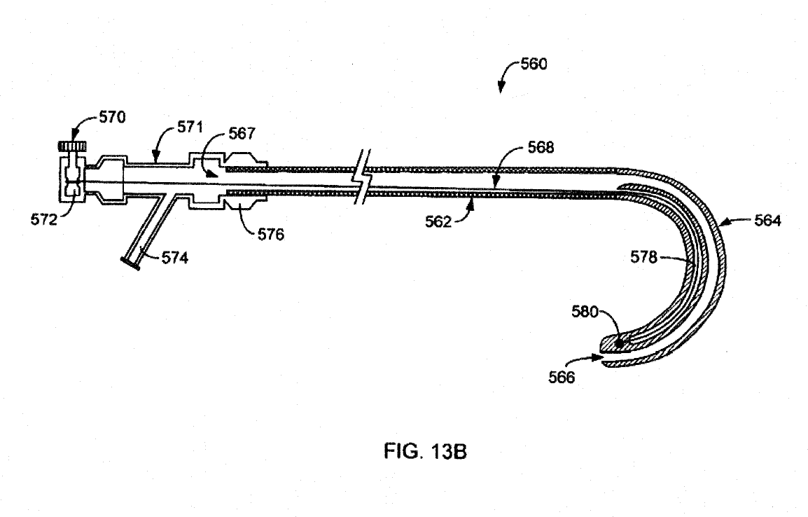

With reference now to FIGS. 13A and 13B, one variation of a steerable catheter device 560 is shown. Steerable catheter device 560 may be used in a method such as that just described in reference to FIGS. 12A-12F, for example in performing a function similar to that performed by second guide catheter 554. In other variations, catheter device 560 may perform any other suitable function. As shown, catheter device 560 suitably includes an elongate catheter body having a proximal portion 562 and a distal portion 564. At least one tensioning member 568, such as but not limited to a tensioning cord, extends from proximal portion 562 to distal portion 564 and is coupled with the distal portion 564 and at least one tensioning actuator 570/572 on the proximal portion. Tensioning actuator 570/572 may include, for example, a knob 570 and a barrel 572 for wrapping and unwrapping tensioning member 568 to apply and remove tension. Tensioning member 568 is coupled with distal portion 564 at one or more connection points 580. In some variations, catheter device 560 includes a proximal housing 571, handle or the like, coupled to the proximal end of proximal portion 562 via a hub 576 or other means. Housing 571 may be coupled with tensioning actuator 570/572 and may include one or more arms 574 for infusing fluid or for other functions. In the variation shown, arm 574 and housing 571 include a lumen 567 that is in fluid communication with a fluid lumen 566 of the catheter body. Fluid may be introduced through arm 574 to pass through fluid lumen 566 to provide, for example, for contrast material at the distal tip of catheter device 560 to enhance visualization of device 560 during a procedure. Any other suitable fluid(s) may be passed through lumens 567/566 for any other purpose. Another lumen 578 may be included in distal portion 564, through which tensioning member 568 passes before attaching at a distal location along distal portion 564.

FIG. 13B shows catheter device 560 in a deformed/bent configuration, after tension has been applied to distal portion 564 by applying tension to tensioning member 568, via knob 570 and barrel 572. The bend in distal portion 564 will allow it to conform more readily to a valve annulus, while catheter device 560 in its straight configuration will be more amenable to passage through vasculature of the patient. Tensioning member 568 may be manufactured from any suitable material or combination of materials, such as but not limited to Nitinol, polyester, nylon, polypropylene and/or other polymers. Some variations may include two or more tensioning members 568 and/or two or more tensioning actuators 570/572 to provide for changes in shape of distal portion 564 in multiple directions. In alternative variations, knob 570 and barrel 572 may be substituted with any suitable devices, such as a pull cord, button, lever or other actuator. Various alternatives may also be substituted for tensioning member 568 in various variations. For example, shaped expandable members, shape memory members and/or the like may be used to change the shape of distal portion 564.

Generally, proximal portion 562 of the catheter body is less flexible than distal portion 564. Proximal portion 562 may be made of any suitable material, such as PEBAX, FEP, nylon, polyethylene and/or the like, and may include a braided material, such as stainless steel, to provide stiffness and strength. Distal portion 564 may be made of similar or other materials, but the braided material is typically not included, to provide for greater flexibility. Both proximal and distal portions 562/564 may have any suitable lengths, diameters, overall configurations and the like. In one variation the catheter body is approximately 140 cm in length and 6 French in diameter, but any other suitable sizes may be used in other variations. Either proximal portion 562, distal portion 564 or preferably both, may be made from or coated with one or more friction resistant or lubricating material to enhance passage of device 560 through an introducer catheter and/or to enhance passage of a sheath or other device over catheter device 560.

Although the foregoing is a complete and accurate description of the present invention, the description provided above is for exemplary purposes only, and variations may be made to the variations described without departing from the scope of the invention. Thus, the above described should not be construed to limit the scope of the invention as described in the appended claims.

What is claimed is:

1. An anchor device comprising:
a catheter; and
a self-securing anchor coupled to a tether, the anchor having an initial constrained undeployed configuration wherein the anchor is constrained by the catheter, and a deployed configuration wherein the anchor is released from the catheter, wherein the anchor has two legs having ends and wherein the legs of the anchor in both the initial undeployed and the deployed configurations cross in a single turning direction from one end to the other end to form a loop, the loop defining an opening that shortens as the anchor changes from the initial undeployed configuration to the deployed configuration, and wherein the leg ends in the deployed configuration curve back toward the loop.

2. The anchor of claim 1 wherein the ends of the legs are blunt.

3. The anchor of claim 1, wherein the ends of the legs are sharp.

4. The anchor of claim 1, wherein the anchor is made of a shape-memory material.

5. The anchor of claim 4, wherein the anchor comprises Nickel-Titanium Alloy.

6. The anchor of claim 1, wherein the anchor is made of a superelastic material.

7. The anchor of claim 1, wherein the legs are collapsed in the undeployed configuration and expanded in the deployed configuration.

8. The anchor of claim 1, wherein, when the anchor is deployed in a tissue, the anchor may expand or collapse from the deployed configuration to absorb energy during dynamic loading of the tissue.

9. The anchor of claim 1, wherein at least a portion of the loop comprises a loop size limiting region that is less flexible than the legs.

10. A method of securing an anchor to tissue comprising:
compressing a self-securing anchor coupled to a tether within a catheter, the anchor having an initial constrained undeployed configuration wherein the anchor is constrained by the catheter, wherein the anchor is compressed into the initial undeployed configuration, the anchor comprising two legs having ends, the legs crossing in a single turning direction from one end to the other end to form a loop, the loop defining an opening; and
releasing the anchor from the initial undeployed configuration into an expanded deployed configuration wherein the anchor is released from the catheter and the opening shortens as the anchor changes from the initial undeployed configuration to the deployed configuration, and wherein the leg ends deploy through the tissue in a curved path toward the loop to secure the anchor into the tissue.

11. The anchor of claim 1, wherein at least a portion of the anchor is coated with an agent.

12. The anchor of claim 11 wherein the agent is selected from the group consisting of an anti-inflammatory agent, an anti-coagulant agent, an anti-proliferative agent, and a pro-proliferative agent.

13. The anchor of claim 12 wherein the agent is a pro-proliferative agent.

14. The anchor of claim 1, wherein at least a portion of the anchor has a region of increased friction.

15. The anchor of claim 1, wherein the anchor comprises at least one sensor.

16. A self-securing anchor coupled to a tether, the anchor having an initial constrained undeployed configuration and a deployed configuration, wherein the anchor has two legs having ends and wherein the legs of the anchor in both the initial undeployed and deployed configurations cross in a single turning direction from one end to the other end to form a loop, and wherein the ends of the legs in the deployed configuration curve back toward the loop.

17. The method of claim 10, wherein the anchor is released into cardiac tissue.

18. The method of claim 10, wherein more than one anchor is released into tissue.

19. The method of claim 10, wherein the anchor is released into tissue of a hollow body organ.
